SEQUENCE LISTING (1 sequence)

SEQ ID NO: 1 - DNA, 24 bp, Artificial Sequence
  Synthetic
  misc_feature (1)..(24): may be modified by phosphorothioate internucleotide linkage
tcgtcgttttgtcgttttgtcgtt

(19) United States
(12) Patent Application Publication
(10) Pub. No.: US 2022/0088059 A1
(43) Pub. Date: Mar. 24, 2022

(54) COMBINED SPHERICAL NUCLEIC ACID AND CHECKPOINT INHIBITOR FOR ANTITUMOR THERAPY

(71) Applicant: Exicure Operating Company, Chicago, IL (US)

(72) Inventors: Pinal Patel, Chicago, IL (US); Weston Daniel, Chicago, IL (US); Alice Bexon, Montclair, NJ (US); Matthias Schroff, Chicago, IL (US)

(73) Assignee: Exicure Operating Company, Chicago, IL (US)

(21) Appl. No.: 17/430,277

(22) PCT Filed: Feb. 12, 2020
(86) PCT No.: PCT/US2020/017999

§ 371 Date: Aug. 11, 2021

Related U.S. Application Data
(60) Provisional application No. 62/946,380, filed on Dec. 10, 2019.
(60) Provisional application No. 62/804,748, filed on Feb. 12, 2019.

(51) Int. Cl. A61K 31/7125; A61K 39/395; A61P 35/00

(52) U.S. Cl. CPC A61K 31/7125; A61K 2039/505; A61P 35/00; A61K 39/3955

(57) ABSTRACT

Aspects of the invention are directed to methods of treating cancer using a combination of spherical nucleic acids (SNAs) and a checkpoint inhibitor. The SNA molecule comprises a core oligonucleotide shell of CpG oligonucleotides positioned on the exterior of this core. The SNA is administered at a fixed dose of 2mg to a solid tumour or tumour lesion, and is administered within 24 hours of the checkpoint inhibitor administration. The SNA can also be administered at a dose of 700 mg-900 mg every 2 weeks. The SNA can also be a CpG linked via a spacer to the exterior surface of a liposome core of 40 nm or less diameter.

### RELATED APPLICATIONS

This application is a national stage filing under 35 U.S.C. § 371 of International PCT Application No. PCT/US2020/017999, filed Feb. 12, 2020, which claims the benefit of the filing dates under 35 U.S.C. § 119(e) of U.S. Provisional Application Ser. No. 62/946,380, filed Dec. 10, 2019 and U.S. Provisional Application Ser. No. 62/804,748, filed Feb. 12, 2019, the entire contents of each of which are incorporated herein by reference in their entireties.

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Aug. 10, 2021, is named A110770020US02-SEQ-DQB and is 730 bytes in size.

### BACKGROUND

Spherical nucleic acid (SNA) constructs are three-dimensional arrangements of oligonucleotides that utilize scavenger receptors to enter cells; this contrasts with other oligonucleotide delivery systems, such as those utilizing cationic lipids or polymers, which often disrupt anionic cell membranes to deliver payloads. Immune checkpoints are inhibitory pathways in the immune system that are crucial for both maintaining self-tolerance and for modulating the duration and amplitude of physiological immune responses. These pathways are critical in minimizing collateral tissue damage by the immune system.

### SUMMARY

Tumors use certain immune-checkpoint pathways as a major mechanism of immune resistance, particularly against T cells that are specific for tumor antigens. These checkpoint pathways can prevent a latent immune response from acting on the tumor. Because many of the immune checkpoints are initiated by ligand-receptor interactions, they can be readily blocked by antibodies or modulated by recombinant forms of ligands or receptors.

According to some aspects, methods for treating cancer using SNAs and/or checkpoint inhibitors are provided herein. In some embodiments, the method for treating cancer comprises administering to a subject a SNA and a checkpoint inhibitor, wherein the SNA comprises a core having an oligonucleotide shell comprised of CpG oligonucleotides positioned on the exterior of the core, wherein the SNA is administered to the subject at a fixed dose of at least about 2 mg to one solid tumor or tumor lesion or divided among two or more solid tumors or tumor lesions in the subject, wherein the SNA is administered within 24 hours of administration of the checkpoint inhibitor, to treat the cancer in the subject.

According to some aspects, the method for treating cancer comprises administering to a subject a SNA and a checkpoint inhibitor, wherein the SNA comprises a core having an oligonucleotide shell comprised of CpG oligonucleotides positioned on the exterior of the core, wherein the SNA is administered at a dose of between 2 mg and 32 mg once a week or every three weeks, or at a dose of between 700 mg and 900 mg every two weeks, wherein the checkpoint inhibitor is administered at a dose of between 180 mg and 370 mg or of between 180 mg and 220 mg every three weeks, wherein the SNA is administered within 24 hours of the administration of the checkpoint inhibitor, and wherein the SNA and the checkpoint inhibitor are administered through different routes of administration to treat the cancer in the subject.

According to some aspects, the method for treating cancer comprises administering a therapeutic dose of a checkpoint inhibitor and a therapeutic dose of a SNA comprising a CpG oligonucleotide linked through a spacer to an exterior surface of a liposome core having a mean diameter of less than 40 nm, wherein the SNA is administered by intratumoral injection into one tumor lesion or into multiple lesions at a dose of between 2 mg and 32 mg and wherein the checkpoint inhibitor is administered by intravenous injection at a dose of between 180 mg and 370 mg or of between 180 mg and 220 mg.

In some embodiments, the SNA is administered subcutaneously or intratumorally to a solid tumor and the checkpoint inhibitor is administered by intravenous infusion.

In some embodiments, the cancer in the subject is not responsive to treatment with the checkpoint inhibitor alone or wherein the cancer in the subject is resistant to treatment with the checkpoint inhibitor alone.

In some embodiments, the subject has not received a small molecule or tyrosine kinase inhibitor within two weeks or five half-lives (whichever is longer) prior to the first dose of the SNA, has not received chemotherapy within 3 weeks prior to the first dose of the SNA, has not received biological cancer therapy within 3 weeks prior to the first dose of the SNA, has not received nitrosourea or radioisotope within 6 weeks prior to first dose of the SNA, has not recovered from an adverse event (G1) or has not been identified as experiencing an adverse event due to cancer therapeutics administered more than 4 weeks prior to the first dose of the SNA.

In some embodiments, the SNA is administered at a dose of between 1 and 3 mg, the SNA is administered at a dose between 3 and 5 mg, the SNA is administered at a dose between 5 and 7 mg, the SNA is administered at a dose between 7 and 9 mg, the SNA is administered at a dose between 9 and 14 mg, the SNA is administered at a dose between 15 and 17 mg, the SNA is administered at a dose between 18 mg and 31 mg, the SNA is administered at a dose between 31 mg and 33 mg, the SNA is administered at a dose between 0.5 mg and 2 mg, the SNA is administered at a dose between 2 and 4 mg, the SNA is administered at a dose between 11 and 13 mg, the SNA is administered at a dose between 23 and 25 mg, the SNA is administered at a dose between 2 and 31 mg, the SNA is administered at a dose between 2 and 30 mg, the SNA is administered at a dose between 2 and 29 mg, the SNA is administered at a dose between 2 and 28 mg, the SNA is administered at a dose between 2 and 27 mg, the SNA is administered at a dose between 2 and 26 mg, the SNA is administered at a dose between 2 and 25 mg, the SNA is administered at a dose between 2 and 24 mg, the SNA is administered at a dose between 2 and 23 mg, the SNA is administered at a dose between 2 and 22 mg, the SNA is administered at a dose between 2 and 21 mg, the SNA is administered at a dose between 2 and 20 mg, the SNA is administered at a dose between 2 and 19 mg, the SNA is administered at a dose between 2 and 18 mg, the SNA is administered at a dose between 2 and 17 mg, the SNA is administered at a dose between 2 and 16 mg, the SNA is administered at a dose between 2 and 15 mg, the SNA is administered at a dose between 2 and 14 mg, the SNA is administered at a dose between 2 and 13 mg, the SNA is administered at a dose between 2 and 12 mg, the SNA is administered at a dose between 2 and 11 mg, the SNA is administered at a dose between 2 and 10 mg, the SNA is administered at a dose between 2 and 9 mg, the SNA is administered at a dose between 2 and 8 mg, the SNA is administered at a dose between 2 and 7 mg, the SNA is administered at a dose between 2 and 6 mg, the SNA is administered at a dose between 2 and 5 mg, the SNA is administered at a dose between 2 and 3 mg, the SNA is administered at a dose of 1 mg, the SNA is administered at a dose of 2 mg, the SNA is administered at a dose of 3 mg, the SNA is administered at a dose of 4 mg, the SNA is administered at a dose of 6 mg, the SNA is administered at a dose of 8 mg, the SNA is administered at a dose of 12 mg, the SNA is administered at a dose of 16 mg, wherein the SNA is administered at a dose of 24 mg, wherein the SNA is administered at a dose of 32 mg.

In some embodiments, the checkpoint inhibitor is administered at a dose between 50 mg and 1000 mg, the checkpoint inhibitor is administered at a dose between 50 mg and 750 mg, the checkpoint inhibitor is administered at a dose between 50 mg and 500 mg, the checkpoint inhibitor is administered at a dose between 50 mg and 400 mg, the checkpoint inhibitor is administered at a dose between 50 mg and 300 mg, the checkpoint inhibitor is administered at a dose between 50 mg and 290 mg, the checkpoint inhibitor is administered at a dose between 50 mg and 280 mg, the checkpoint inhibitor is administered at a dose between 50 mg and 270 mg, the checkpoint inhibitor is administered at a dose between 50 mg and 260 mg, the checkpoint inhibitor is administered at a dose between 50 mg and 250 mg, the checkpoint inhibitor is administered at a dose between 50 mg and 240 mg, the checkpoint inhibitor is administered at a dose between 50 mg and 230 mg, the checkpoint inhibitor is administered at a dose between 50 mg and 220 mg, the checkpoint inhibitor is administered at a dose between 50 mg and 210 mg, the checkpoint inhibitor is administered at a dose between 50 mg and 200 mg, the checkpoint inhibitor is administered at a dose between 60 mg and 200 mg, the checkpoint inhibitor is administered at a dose between 70 mg and 200 mg, the checkpoint inhibitor is administered at a dose between 80 mg and 200 mg, the checkpoint inhibitor is administered at a dose between 90 mg and 200 mg, the checkpoint inhibitor is administered at a dose between 100 mg and 200 mg, the checkpoint inhibitor is administered at a dose between 110 mg and 200 mg, the checkpoint inhibitor is administered at a dose between 120 mg and 200 mg, the checkpoint inhibitor is administered at a dose between 130 mg and 200 mg, the checkpoint inhibitor is administered at a dose between 140 mg and 200 mg, the checkpoint inhibitor is administered at a dose between 150 mg and 200 mg, the checkpoint inhibitor is administered at a dose between 160 mg and 200 mg, the checkpoint inhibitor is administered at a dose between 170 mg and 200 mg, the checkpoint inhibitor is administered at a dose between 180 mg and 200 mg, the checkpoint inhibitor is administered at a dose between 190 mg and 200 mg, the checkpoint inhibitor is administered at a dose of 200 mg.

In some embodiments, the cancer is biliary tract cancer, brain cancer, breast cancer, cervical cancer, choriocarcinoma, colon cancer, endometrial cancer, esophageal cancer, gastric cancer, an intraepithelial neoplasm, leukemia, lymphoma, liver cancer, lung cancer, melanoma, neuroblastoma, oral cancer, ovarian cancer, pancreatic cancer, pancreatic adenocarcinoma, prostate cancer, hormone refractory prostate adenocarcinoma, rectal cancer, sarcomas, testicular cancer, thyroid cancer, anaplastic thyroid cancer, renal cancer, hairy cell leukemia, chronic myelogenous leukemia, cutaneous T-cell leukemia, multiple myeloma, renal cell carcinoma, clear cell renal cell carcinoma, bladder cancer, non-small cell lung cancer (NSCLC), glioma, or glioblastoma multiforme.

In some embodiments, the cancer is Merkel cell carcinoma, cutaneous squamous cell carcinoma, melanoma or squamous cell carcinoma of the head and neck.

In some embodiments, the cancer is a sarcoma, including pleomorphic sarcoma, gastrointestinal stromal tumor (GIST), liposarcoma, leiomyosarcoma, synovial sarcoma, malignant peripheral nerve sheath tumor, rhabdomyosarcoma, angiosarcoma, fibrosarcoma, dermatofibrosarcoma protuberans, epithelioid sarcoma, myxoma, mesenchymoma, vascular sarcoma, neurilemmoma, bone sarcoma, osteosarcoma, Ewing's sarcoma, chondrosarcoma, Kaposi sarcoma, solitary fibrous tumor, chordoma, desmoid-type fibromatosis, fibroblastic sarcoma, giant cell tumor of the bone, gynaecological sarcoma, soft tissue sarcoma, angioleiomyoma, leiomyoma, smooth muscle sarcoma, fibrohistiocytic sarcoma.

In some embodiments, the subject has a solid tumor or a tumor lesion that can be injected intratumorally via one or more of palpation or ultrasound.

In some embodiments, the solid tumor or tumor lesion is on or near the skin, on or near cutaneous soft tissue, on or near subcutaneous soft tissue, and/or in or near a lymph node.

In some embodiments, the SNA is administered to one or more of a cutaneous tumor lesion, a subcutaneous tumor lesion or a nodal lesion.

In some embodiments, the SNA is further administered to one or more deep visceral lesion.

In some embodiments, the deep visceral lesion is in an internal organ of the body of the subject.

In some embodiments, the deep visceral lesion is in the liver, heart, pancreas, kidney, stomach, lung, colon, or intestines.

In some embodiments, the core is about 15 nm to about 30 nm in mean diameter. In some embodiments, the core, such as a liposomal core or liposome core, of a SNA disclosed herein has a mean diameter of about 10 to about 150 nm. In some embodiments, the mean diameter of the core is from about 15 nm to about 100 nm, about 20 nm to about 100 nm, about 25 nm to about 100 nm, about 15 nm to about 50 nm, about 20 nm to about 50 nm, about 10 nm to about 70 nm, about 15 nm to about 70 nm, about 20 nm to about 70 nm, about 10 nm to about 30 nm, about 15 nm to about 30 nm, about 20 nm to about 30 nm, about 10 nm to about 40 nm, about 15 nm to about 40 nm, about 20 nm to about 40 nm, about 10 nm to about 80 nm, about 15 nm to about 80 nm, or about 20 nm to about 80 nm.

In some embodiments, the core is about 15 nm to about 30 nm in diameter. In some embodiments, the core, such as a liposomal core or liposome core, of a SNA disclosed herein has a diameter of about 10 to about 150 nm. In some embodiments, the diameter of the core is from about 15 nm to about 100 nm, about 20 nm to about 100 nm, about 25 nm to about 100 nm, about 15 nm to about 50 nm, about 20 nm to about 50 nm, about 10 nm to about 70 nm, about 15 nm to about 70 nm, about 20 nm to about 70 nm, about 10 nm to about 30 nm, about 15 nm to about 30 nm, about 20 nm to about 30 nm, about 10 nm to about 40 nm, about 15 nm to about 40 nm, about 20 nm to about 40 nm, about 10 nm to about 80 nm, about 15 nm to about 80 nm, or about 20 nm to about 80 nm.

In some embodiments, the core, such as a liposomal core or liposome core, of a SNA disclosed herein has a mean diameter of about or less than about 10 nm, 15 nm, 20 nm, 25 nm, 30 nm, 35 nm, and/or 40 nm.

In some embodiments, the CpG oligonucleotides comprise a spacer.

In some embodiments, the spacer is or comprises oligoethylene glycol.

In some embodiments, the oligoethylene glycol is a hexaethylene glycol.

In some embodiments, the SNA has about 25 to 35 CpG oligonucleotides positioned on the exterior surface of the core.

In some embodiments, the CpG oligonucleotides comprise the nucleotide sequence of CpG-7909. In some embodiments, the CpG oligonucleotides comprise the nucleotide sequence 5′-TCGTCGTTTTGTCGTTTTGTCGTT-3′(SEQ ID NO: 1).

In some embodiments, the CpG oligonucleotides comprise the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1) wherein * represents phosphorothioate internucleotide linkage, HEG represents Hexa(ethylene glycol)phosphodiester, and TEG Cholesteryl Ester represents (N-cholesteryl-3-aminopropyl)-triethyleneglycol-glyceryl-1-O-phosphodiester.

In some embodiments, the checkpoint inhibitor is a PD-1 antibody or a PD-L1 antibody. In some embodiments, the checkpoint inhibitor is pembrolizumab or cemiplimab.

In some embodiments, the cancer in the subject is refractory or resistant to treatment with a checkpoint inhibitor. In some embodiments, the cancer in the subject is refractory or resistant to treatment with pembrolizumab. In some embodiments, the cancer in the subject is refractory or resistant to treatment with cemiplimab. In some embodiments, the cancer in the subject is refractory or resistant to treatment with nivolumab. In some embodiments, the cancer in the subject is refractory or resistant to treatment with avelumab.

In some embodiments, the SNA is administered within 24 hours of administration of the checkpoint inhibitor. In some embodiments, the SNA is administered within 12 hours of administration of the checkpoint inhibitor.

In some embodiments, the liposome core is less than 30 nm in mean diameter. In some embodiments, the liposome core is about 15 nm to 40 nm in mean diameter.

In some embodiments, the SNA and the checkpoint inhibitor are administered substantially at the same time.

In some embodiments, the checkpoint inhibitor is administered at a dose between 50 mg and 350 mg.

In some embodiments, the checkpoint inhibitor is administered at a dose between 50 mg and 1200 mg.

In some embodiments, the checkpoint inhibitor is administered at a dose between 1 mg/kg and 10 mg/kg.

In some embodiments, the SNA is administered prior to administration of the checkpoint inhibitor.

In some embodiments, the SNA is administered after the administration of the checkpoint inhibitor.

In some embodiments, the method for treating cancer comprises administering to a subject a spherical nucleic acid (SNA) and a checkpoint inhibitor, wherein the SNA comprises a core having an oligonucleotide shell comprised of CpG oligonucleotides positioned on the exterior of the core, wherein the SNA is administered to the subject at a fixed dose of between about 16 mg to about 32 mg to one solid tumor or tumor lesion or at a fixed dose of between about 16 mg to about 32 mg divided among two or more solid tumors or tumor lesions in the subject, wherein the SNA is administered within 24 hours of administration of the checkpoint inhibitor, to treat the cancer in the subject, wherein the cancer is Merkel cell carcinoma or cutaneous squamous cell carcinoma.

In some embodiments, the method for treating cancer comprises administering to a subject a spherical nucleic acid (SNA) and a checkpoint inhibitor, wherein the SNA comprises a core having an oligonucleotide shell comprised of CpG oligonucleotides positioned on the exterior of the core, wherein the SNA is administered at a dose of between about 16 mg to about 32 mg once a week, wherein the checkpoint inhibitor is administered at a dose of between 180 and 370 mg or of between 180 mg and 220 mg every three weeks or at a dose of between 700 mg and 900 mg every two weeks, wherein the SNA is administered within 24 hours of the administration of the checkpoint inhibitor, and wherein the SNA and the checkpoint inhibitor are administered through different routes of administration to treat the cancer in the subject, wherein the cancer is Merkel cell carcinoma or cutaneous squamous cell carcinoma.

In some embodiments, the method for treating cancer comprises administering a therapeutic dose of a spherical nucleic acid (SNA) comprising a CpG oligonucleotide linked through a spacer to an exterior surface of a liposome core having a diameter of less than about 40 nm and a checkpoint inhibitor, wherein the SNA is administered by intratumoral injection into multiple lesions at a dose of between about 16 mg and about 32 mg and the checkpoint inhibitor is administered by intravenous injection at a dose of between 180 and 370 mg or between 700 mg and 900 mg, wherein the cancer is Merkel cell carcinoma or cutaneous squamous cell carcinoma.

In some embodiments, the SNA is administered at a dose of or about 16 mg. In some embodiments, the SNA is administered at a dose of or about 32 mg. In some embodiments, wherein the SNA is administered at a dose of or about 16 mg and wherein the checkpoint inhibitor is administered at a dose of or about 200 mg. In some embodiments, the SNA is administered at a dose of or about 32 mg and wherein the checkpoint inhibitor is administered at a dose of or about 200 mg.

In some embodiments, the checkpoint inhibitor is pembrolizumab administered at a dose of or about 200 mg and the cancer is Merkel cell carcinoma. In some embodiments, the SNA is administered at a dose of or about 16 mg and wherein the checkpoint inhibitor is administered at a dose of or about 350 mg. In some embodiments, the SNA is administered at a dose of or about 32 mg and wherein the checkpoint inhibitor is administered at a dose of or about 350 mg. In some embodiments, the checkpoint inhibitor is cemiplimab administered at a dose of or about 350 mg and wherein the cancer is cutaneous squamous cell carcinoma. In some embodiments, the checkpoint inhibitor is avelumab administered at a dose of or about 800 mg and the cancer is Merkel cell carcinoma. In some embodiments, the SNA is administered at a dose of or about 16 mg and wherein the checkpoint inhibitor is administered at a dose of or about 800 mg. In some embodiments, the SNA is administered at a dose of or about 32 mg and wherein the checkpoint inhibitor is administered at a dose of or about 800 mg.

In some embodiments, the administration of the SNA or the SNA in combination with the checkpoint inhibitor results in one or more of increased cytokine expression, increased chemokine expression, or increased immune cell activation by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, relative to a reference level. In some embodiments, the administration of the SNA or the SNA in combination with the checkpoint inhibitor results in one or more of increased cytokine expression, increased chemokine expression, or increased immune cell activation by at least or about 100%, at least or about 150%, at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 15-fold, at least or about 20-fold, at least or about 30-fold, at least or about 40-fold, at least or about 50-fold or more, relative to a reference level.

In some embodiments, the cancer in the subject is progressive disease and administration of the SNA or administration of the SNA in combination with the checkpoint inhibitor for the treatment of the cancer in the subject renders the cancer stable disease. In some embodiments, administration of the SNA or administration of the SNA in combination with the checkpoint inhibitor for the treatment of the cancer in the subject facilitates partial response or complete response of the cancer.

In some embodiments, the cancer is stable disease for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks. In some embodiments, the cancer is stable disease for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months. In some embodiments, the cancer is stable disease for at least one year, at least two years, at least three years, at least four years, at least five years, at least six years, at least seven years, at least eight years, at least nine years, at least 10 years, at least 11 years or at least 12 years.

In some embodiments, the treatment results in partial response or complete response for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks in the subject. In some embodiments, the treatment results in partial response or complete response for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months in the subject. In some embodiments, the treatment results in partial response or complete response for at least one year, at least two years, at least three years, at least four years, at least five years, at least six years, at least seven years, at least eight years, at least nine years, at least 10 years, at least 11 years or at least 12 years in the subject.

In some embodiments, the subject has at least one target lesion, at least two target lesions, at least three target lesions or at least four target lesions and wherein administration of the SNA or the SNA in combination with the checkpoint inhibitor decreases the diameter of at least one target lesion in the subject or decreases the sum of the diameters of two or more target lesions in the subject by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99% relative to a reference level. In some embodiments, the subject has at least one target lesion, at least two target lesions, at least three target lesions or at least four target lesions and wherein administration of the SNA or the SNA in combination with the checkpoint inhibitor results in partial response or results in complete response in at least one target lesion, at least two target lesions, at least three target lesions, or at least four target lesions in the subject.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. For purposes of clarity, not every component may be labeled in every drawing. In the drawings:

FIG. 1 shows an overview of Study Design.

FIG. 2 shows monotherapy dose escalation rules.

FIG. 3 shows combination therapy dose escalation rules.

FIG. 4 shows individual patient decision making rules.

FIG. 5 shows total dose volume algorithm.

FIG. 6 shows CpG-SNA tumor administration technique. Cycle 1 (CpG-SNA monotherapy) will be 2 weeks long and comprise intratumoral (IT) injections on days 1 and 8. For Cycles 2 and 3, CpG-SNA will be administered on a weekly basis, on days 1, 8, and 15 of each 3-week cycle. Thereafter (Cycle 4+), CpG-SNA will be administered every 3 weeks on day 1 until lack of clinical benefit or disease progression.

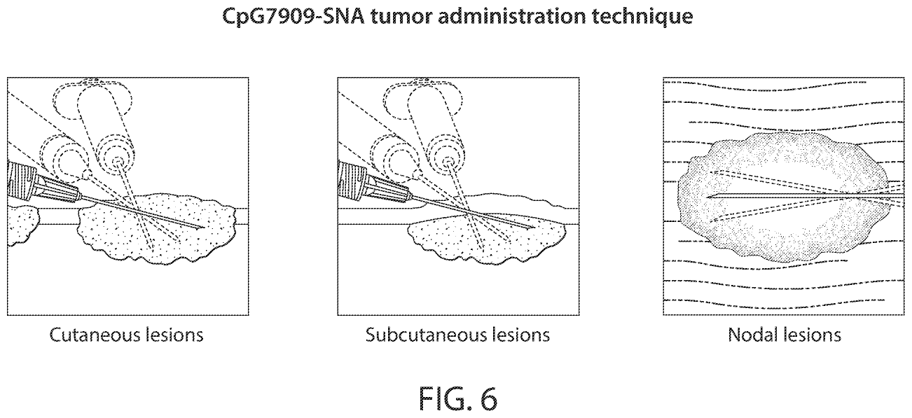

FIG. 7 shows an overview of Study Design with Phase 2 expansion in Merkel cell carcinoma (MCC) and in cutaneous squamous-cell carcinoma (CSCC)

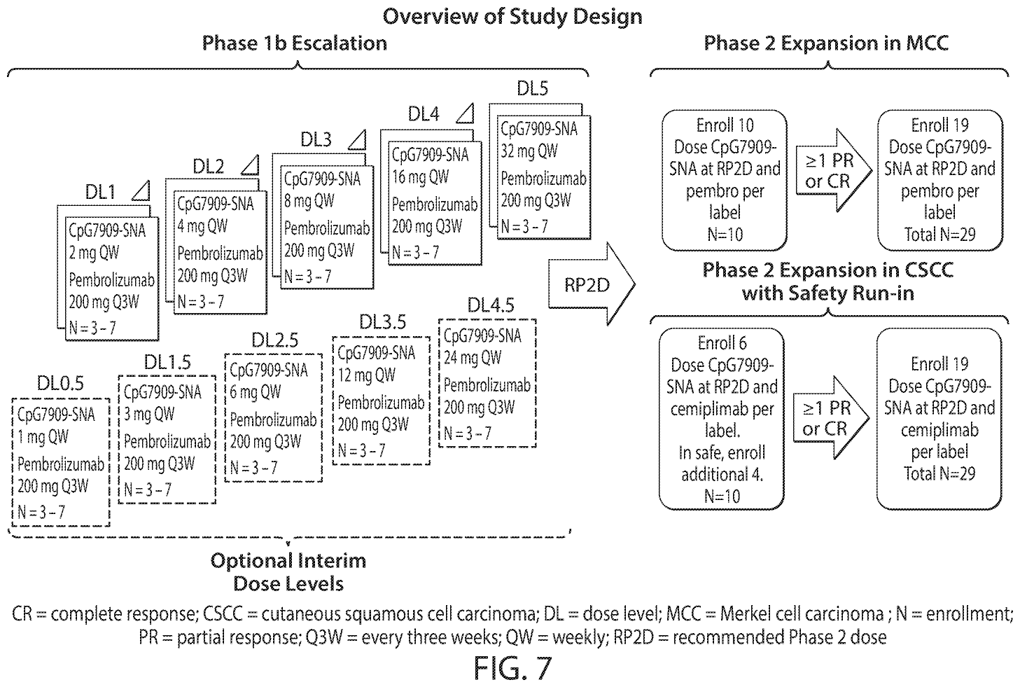

FIG. 8 shows dosing and assessment schedule.

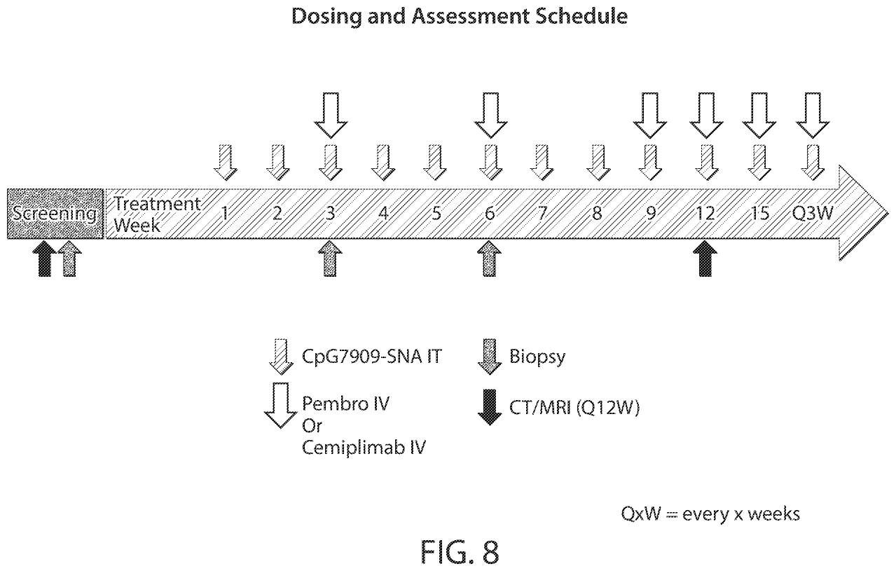

### DETAILED DESCRIPTION

Antibodies targeting cytotoxic T-lymphocyte-associated protein 4 (CTLA-4), programmed death 1 (PD-1), and programmed death ligand 1 (PD-L1) are used for treatment of advanced melanoma, head and neck squamous cell carcinoma, classic Hodgkin's lymphoma, renal cell carcinoma, urothelial carcinoma, cutaneous squamous cell carcinoma, Merkel cell carcinoma, and non-small cell lung cancer. However, despite the promise of these therapies, there is room to improve response rates to these drugs. For example, the PD-1 inhibitor nivolumab produced response rates of up to 40% in advanced melanoma trials [Opdivo® USPI]. The use of a spherical nucleic acid (SNA) disclosed herein, such as CpG-SNA, which is comprised of CpG oligonucleotides that are agonists of the toll-like receptor (TLR) 9, increases the amplitude of the immune response against tumors while simultaneously inhibiting the immune checkpoints with an inhibitor antibody. The activation of the TLR9 by a SNA disclosed herein produces immune responses that are useful in oncology applications. TLR9 stimulation initially activates antigen-nonspecific innate immunity followed by antigen-specific adaptive immunity.

CpG7909-SNA is a novel SNA configuration of a TLR9 agonist oligonucleotide, designed to trigger innate and adaptive immune responses in patients with cancer. CpG7909-SNA is intended to be administered intratumorally (IT) for the treatment of solid tumors.

In vitro experiments show that CpG-SNA exhibits cellular uptake and activity that is greater than the corresponding linear oligonucleotide and demonstrates specificity for TLR9. CpG-SNA has the potential to treat a variety of tumor types as shown by the decreased tumor volume and increased median survival of exposed animals compared to vehicle controls. Further, CpG-SNA shows anti-tumor activity after administration by any of three routes—the subcutaneous (SC), intratumoral (IT), or intravenous (IV) routes. Finally, across a variety of tumor models and routes of administration, a CpG-SNA can be combined with checkpoint inhibitor (CPI) antibodies, such as anti-PD-1 or anti-PD-L1, to produce enhanced anti-tumor effects compared with either agent alone.

Anti-PD-1 antibody treatment blocks the interaction of PD-1 and the upregulated PD-L1 on tumor-targeting T cells and tumor cells, respectively, to ensure that the anti-tumor T cells are not inactivated. However, anti-PD-1 antibody refractory/non-responding patients may not have tumor specific T cells to act on the cancer tissue. TLR9 agonism within the tumor is designed to stimulate tumor cell killing by NK cells, stimulate tumor antigen generation, and eventually stimulate activation of tumor specific cytotoxic T cells, thus addressing the lack of tumor-specific T cells in the patients. In patients where tumor specific T cells are already present, CPIs may work as a monotherapy, but the response can be improved by pushing the immune system further by its activation with a TLR9 agonist, as disclosed herein.

SNAs, such as CpG-SNAs disclosed herein, exhibit a suite of mechanistic and functional properties that make it ideal for agonizing TLR9. First, SNAs are more efficiently taken into and concentrated in endosomes compared to linear oligonucleotides (i.e., oligonucleotides not in the SNA configuration). Second, the oligonucleotides delivered as a part of SNAs cause an enhanced cytokine response, both in magnitude and duration, compared to linear oligonucleotides. Third, the SNA projects its oligonucleotides outward, allowing it to act upon TLRs. This arrangement of nucleic acids or oligonucleotides in the SNA is in contrast to other oligonucleotide delivery systems where the oligonucleotides are held, for instance, internally inside virus-like particles. Taken together, these properties make SNAs, such as CpG-SNAs, effective for an immune-oncology-based therapeutic and an exceptional combination partner for immune-mediated oncology therapeutics, such as checkpoint inhibitor antibodies, including, but not limited to anti-PD-1 antibodies, such as pembrolizumab and cemiplimab, anti-PD-L1 antibodies, such as avelumab, or anti-PD-1 antibodies and anti-PD-L1 antibodies. Disclosed herein are different doses, routes of administration and orders of administration that unexpectedly result in the successful treatment of cancer in humans.

Accordingly, in some embodiments, methods for treating cancer are disclosed herein. In some embodiments, the method for treating cancer comprises administering to a subject a SNA and a checkpoint inhibitor, wherein the SNA comprises a core having an oligonucleotide shell comprised of CpG oligonucleotides positioned on the exterior of the core, wherein the SNA is administered to the subject at a fixed dose of at least about 2 mg. In some embodiments, the fixed dose is administered to one solid tumor or to one tumor lesion in the subject or the fixed dose is divided among two or more solid tumors or two or more tumor lesions in the subject. In some embodiments, the SNA is administered within 24 hours of administration of the checkpoint inhibitor in any order (e.g., the SNA is administered first within the 24-hour period or the checkpoint inhibitor is administered first with the 24-hour period) to treat the cancer in the subject.

A SNA is a three-dimensional arrangement of nucleic acids or oligonucleotides, such as CpG oligonucleotides, comprising an oligonucleotide shell, with densely packed and radially arranged oligonucleotides on the exterior of a core. The SNA is composed of oligonucleotides and a core. The core may be a hollow core which is produced by a three-dimensional arrangement of molecules which form the outer boundary of the core. For instance, the molecules may be in the form of a lipid layer (e.g. lipid monolayer or lipid bilayer), which has a hollow center. Alternatively, the molecules may be in the form of lipids, such as amphipathic lipids (e.g., sterols), which are linked to or associated with, either directly or indirectly, an end of the oligonucleotide. In some embodiments, sterols, such as cholesterol, linked to an end of an oligonucleotide may associate with the outer surface of a core (e.g., a hollow core), such that the oligonucleotides radiate outward from the core. The core may also be a solid or semi-solid core. In some embodiments, the core is a liposomal core.

In some embodiments, the oligonucleotides in the SNA are associated with the core. An oligonucleotide that is associated with the core, such as a liposome core, may be covalently or non-covalently linked to the exterior surface of the core. In some embodiments, an oligonucleotide is associated with a core (e.g., liposomal core) through hydrophobic interactions. For instance, when a sterol is associated with the outer edge of the core, an oligonucleotide may be covalently linked to the sterol directly or indirectly. In some embodiments, the oligonucleotide is covalently linked to the sterol indirectly through a spacer. In some embodiments, when a lipid layer forms the core, the oligonucleotide may be covalently linked to one or more lipids in the lipid layer. In some embodiments, the oligonucleotide may be non-covalently linked to the lipid layer. In some embodiments, the oligonucleotides may be non-covalently linked to the lipid layer by interactions (e.g., hydrophobic interactions) of the oligonucleotide with the lipid layer, or by interactions of a molecule (e.g., a cholesterol) attached to the oligonucleotide, either directly or indirectly (e.g., linked through a spacer), with the lipid layer. In some embodiments, the lipid layer is a lipid bilayer.

In some embodiments, a therapeutic dose of a SNA, a checkpoint inhibitor, or the combination of a SNA and checkpoint inhibitor disclosed herein refers to a dose or dose combination that inhibits tumor cell growth or tumor growth by at least or at least about 5%, at least or at least about 10%, at least or at least about 15%, at least or at least about 20%, at least or at least about 30%, at least or at least about 35%, at least or at least about 40%, at least or at least about 45%, at least or at least about 50%, at least or at least about 55%, at least or at least about 60%, at least or at least about 65%, at least or at least about 70%, at least or at least about 75%, at least or at least about 80%, at least or at least about 85%, at least or at least about 90%, at least or at least about 95%, at least or at least about 99%, relative to a reference level.

In some embodiments, a therapeutic dose of a compound disclosed herein, such as a therapeutic dose of a SNA, a checkpoint inhibitor, or the combination of a SNA with a checkpoint inhibitor disclosed herein, can decrease the number of solid tumors or tumor lesions, decrease the tumor size of one or more of the solid tumors or tumor lesions, or otherwise ameliorate symptoms associated with cancer in a subject. In some embodiments, the tumor size is decreased by at least or at least about 5%, at least or at least about 10%, at least or at least about 15%, at least or at least about 20%, at least or at least about 30%, at least or at least about 35%, at least or at least about 40%, at least or at least about 45%, at least or at least about 50%, at least or at least about 55%, at least or at least about 60%, at least or at least about 65%, at least or at least about 70%, at least or at least about 75%, at least or at least about 80%, at least or at least about 85%, at least or at least about 90%, at least or at least about 95%, at least or at least about 99%, relative to a reference tumor size.

The terms “baseline tumor size” or “reference tumor size,” as disclosed herein, refers to the size of a solid tumor or a tumor lesion, refers to an average of two or more of solid tumors or two or more tumor lesions, or refers to a sum of diameters of two or more of solid tumors or two or more tumor lesions, in a subject with cancer prior to administration of a therapeutic dose of a SNA (e.g., a SNA disclosed herein) only, prior to administration of a therapeutic dose of a checkpoint inhibitor (e.g., a checkpoint inhibitor disclosed herein) only, or prior to administration of a therapeutic dose of a SNA (e.g., a SNA disclosed herein) and a checkpoint inhibitor (e.g., a checkpoint inhibitor disclosed herein). In some embodiments, the baseline tumor size or reference tumor size refers to the baseline sum of diameters of solid tumors or tumor lesions in a subject with cancer.

In some embodiments, amelioration of symptoms associated with cancer refers to amelioration of pain.

In some embodiments, a therapeutic dose results in a certain tumor response, as measured by the Response Evaluation Criteria in Solid Tumors (RECIST) criteria. The RECIST criteria, known to those of ordinary skill in the art, are used to determine objective tumor response for target lesions. (See e.g., Eisenhauer et al. Eur J Cancer (2009) 45(2):228-47). In some embodiments, administration of a therapeutic dose of a SNA, a checkpoint inhibitor, or the combination of a SNA and a checkpoint inhibitor disclosed herein results in a complete response, as measured by the RECIST criteria.

In some embodiments, a “complete response” to a therapeutic dose of a SNA, a checkpoint inhibitor, or the combination of a SNA and a checkpoint inhibitor disclosed herein, as defined by the RECIST criteria and as used herein, refers to the disappearance of all target lesions. In some embodiments, a complete response refers to the disappearance of one or more target lesions (e.g., the disappearance of at least one target lesion, at least two target lesions, at least three target lesions, at least four target lesions, at least five target lesions, at least six target lesions, at least seven target lesions, at least eight target lesions, at least nine target lesions, at least 10 target lesions, at least 11 target lesions, or at least 12 target lesions, etc., or any range or combination thereof). In a “complete response,” pathological lymph nodes (whether target or non-target) have reduction in short axis to less than 10 mm.

In some embodiments, administration of a therapeutic dose of a SNA, a checkpoint inhibitor, or the combination of a SNA and a checkpoint inhibitor disclosed herein results in a “partial response,” as measured by the RECIST criteria and as used herein. A “partial response” to a therapeutic dose of a SNA, a checkpoint inhibitor, or the combination of a SNA and checkpoint inhibitor disclosed herein, as defined by the RECIST criteria and as used herein, refers to at least a 30% decrease in the sum of diameters of target lesions, taking as reference the baseline sum diameters.

In some embodiments, a partial response refers to a decrease in the diameter of at least one target lesion (e.g., a decrease in the diameter of one target lesion, at least two target lesions, at least three target lesions, at least four target lesions, at least five target lesions, at least six target lesions, at least seven target lesions, at least eight target lesions, at least nine target lesions, at least 10 target lesions, at least 11 target lesions, or at least 12 target lesions, etc., or any range or combination thereof) or a decrease in the sum of the diameters of two or more target lesions (e.g., a decrease in the sum of the diameters of at least two target lesions, at least three target lesions, at least four target lesions, at least five target lesions, at least six target lesions, at least seven target lesions, at least eight target lesions, at least nine target lesions, at least 10 target lesions, at least 11 target lesions, or at least 12 target lesions, etc., or any range or combination thereof) by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, relative to a baseline level or a reference level.

The terms “baseline level” or “reference level,” as disclosed herein, are used interchangeably to refer to a corresponding level in a subject with cancer that has been administered a SNA (e.g., a SNA disclosed herein) only, to refer to a corresponding level in a subject with cancer that has been administered a checkpoint inhibitor (e.g., a checkpoint inhibitor disclosed herein) only, or to refer to a corresponding level in a subject with cancer that has not been administered a SNA (e.g., a SNA disclosed herein) or a checkpoint inhibitor (e.g., a checkpoint inhibitor disclosed herein). In some embodiments, a baseline level or reference level refers to a corresponding level in a subject without the corresponding cancer (e.g., a subject without melanoma, Merkel cell carcinoma, cutaneous squamous cell carcinoma, head and neck squamous cell carcinoma, or mucosal melanoma). In some embodiments, a baseline level or reference level refers to a corresponding level in a subject without cancer.

In some embodiments, a baseline level or reference level refers to a corresponding level in a population of cells obtained from a subject with cancer that has been administered a SNA (e.g., a SNA disclosed herein) only, to refer to a corresponding level in a population of cells obtained from a subject with cancer that has been administered a checkpoint inhibitor (e.g., a checkpoint inhibitor disclosed herein) only, or to refer to a corresponding level in a population of cells obtained from a subject with cancer that has not been administered a SNA (e.g., a SNA disclosed herein) or a checkpoint inhibitor (e.g., a checkpoint inhibitor disclosed herein). In some embodiments, a baseline level or reference level refers to a corresponding level in a population of cells obtained from a subject without the corresponding cancer (e.g., a subject without melanoma, Merkel cell carcinoma, cutaneous squamous cell carcinoma, head and neck squamous cell carcinoma, or mucosal melanoma). In some embodiments, a baseline level or reference level refers to a corresponding level in a population of cells obtained from a subject without cancer. In some embodiments, the cancer in the subject is progressive disease and administration of a SNA (e.g., a therapeutic dose of a SNA disclosed herein) or administration of a SNA in combination with a checkpoint inhibitor (e.g., a therapeutic dose of a SNA disclosed herein and a therapeutic dose of a checkpoint inhibitor disclosed herein) for the treatment of the cancer in the subject renders the cancer stable disease.

In some embodiments, the cancer is stable disease for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks. In some embodiments, the cancer is stable disease for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months. In some embodiments, the cancer is stable disease for at least one year, at least two years, at least three years, at least four years, at least five years, at least six years, at least seven years, at least eight years, at least nine years, at least 10 years, at least 11 years or at least 12 years.

In some embodiments, a subject with cancer has at least one target lesion, at least two target lesions, at least three target lesions or at least four target lesions and wherein administration of a SNA (e.g., a therapeutic dose of a SNA disclosed herein) or the SNA in combination with the checkpoint inhibitor (e.g., a therapeutic dose of a SNA disclosed herein and a therapeutic dose of a checkpoint inhibitor disclosed herein) decreases the diameter of at least one target lesion in the subject or decreases the sum of the diameters of two or more target lesions in the subject by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99% relative to a reference level.

In some embodiments, a subject with cancer has at least one target lesion, at least two target lesions, at least three target lesions or at least four target lesions and wherein administration of the SNA (e.g., a therapeutic dose of a SNA disclosed herein) or the SNA in combination with the checkpoint inhibitor (e.g., a therapeutic dose of a SNA disclosed herein and a therapeutic dose of a checkpoint inhibitor disclosed herein) results in partial response or results in complete response in at least one target lesion, at least two target lesions, at least three target lesions, or at least four target lesions in the subject with cancer.

In some embodiments, the treatment (e.g., administration of the SNA, such as a therapeutic dose of a SNA disclosed herein, or administration of the SNA in combination with the checkpoint inhibitor, such as a therapeutic dose of a SNA disclosed herein and a therapeutic dose of a checkpoint inhibitor disclosed herein) results in partial response (e.g., overall partial response) or complete response (e.g., overall complete response) for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks in the subject.

In some embodiments, the treatment (e.g., administration of the SNA, such as a therapeutic dose of a SNA disclosed herein, or administration of the SNA in combination with the checkpoint inhibitor, such as a therapeutic dose of a SNA disclosed herein and a therapeutic dose of a checkpoint inhibitor disclosed herein) results in partial response or complete response for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months in the subject.

In some embodiments, the treatment (e.g., administration of the SNA, such as a therapeutic dose of a SNA disclosed herein, or administration of the SNA in combination with the checkpoint inhibitor, such as a therapeutic dose of a SNA disclosed herein and a therapeutic dose of a checkpoint inhibitor disclosed herein) results in partial response or complete response for at least one year, at least two years, at least three years, at least four years, at least five years, at least six years, at least seven years, at least eight years, at least nine years, at least 10 years, at least 11 years or at least 12 years in the subject.

In some embodiments, a SNA comprises a densely packed oligonucleotide shell with oligonucleotides, such as CpG oligonucleotides, which are radially oriented and stimulate a toll-like receptor (TLR), such as TLR9, resulting in an immune response. In some embodiments, the oligonucleotides in the oligonucleotide shell are oriented radially around a core (e.g., a liposome core or liposomal core).

A CpG oligonucleotide, an immunostimulatory CpG oligonucleotide or an immunostimulatory CpG oligonucleotide refers to any CpG-containing oligonucleotide that is capable of activating an immune cell. At least the C of the CpG dinucleotide is typically unmethylated. Immunostimulatory CpG oligonucleotides are described in a number of issued patents and published patent applications, including U.S. Pat. Nos. 6,194,388; 6,207,646; 6,218,371; 6,239,116; 6,339,068; 6,406,705; and 6,429,199, which are incorporated by reference herein.

In some embodiments, a CpG oligonucleotide is 10, 11, 12, 13, 14, 15, 16, 17, 18, 19, 20, 21, 22, 23, 24, 25, 26, 27, 28, 29, 30, 31, 32, 33, 34, 35 or more than 35 nucleotides in length. In some embodiments, the CpG oligonucleotide is single-stranded. In some embodiments, the CpG oligonucleotide is hybridized to a second oligonucleotide (e.g., a second CpG oligonucleotide) and forms a double-stranded oligonucleotide (e.g., a double-stranded CpG oligonucleotide). In some embodiments, the CpG oligonucleotide is not hybridized to a second oligonucleotide (e.g., a second CpG oligonucleotide) and does not form a double-stranded oligonucleotide (e.g., is not a double-stranded CpG oligonucleotide).

In some embodiments, at least one oligonucleotide (e.g., a CpG oligonucleotide) has its 5′-terminus exposed on the exterior surface away from the core. In some embodiments, all of the oligonucleotides (e.g., CpG oligonucleotides) in a SNA have their 5′-termini exposed on the exterior surface away from the core. In some embodiments, at least one oligonucleotide (e.g., a CpG oligonucleotide) has its 3′-terminus exposed on the exterior surface away from the core. In some embodiments, all of the oligonucleotides (e.g., CpG oligonucleotides) in a SNA have their 3′-termini exposed on the exterior surface (away from the core). In some embodiments, the SNA does not include an oligonucleotide inside the core (e.g., liposome core or liposomal core).

In some embodiments, two or more of the oligonucleotides (e.g., CpG oligonucleotides) in a SNA are crosslinked. In some embodiments, all of the oligonucleotides (e.g., CpG oligonucleotides) in a SNA are crosslinked. In some embodiments, the oligonucleotides (e.g., CpG oligonucleotides) in a SNA are not crosslinked.

In some embodiments the SNA is an agonist of a TLR or a TLR agonist. A TLR agonist, as used herein is a nucleic acid molecule or oligonucleotide that interacts with and stimulates the activity of a TLR. The SNA, in some embodiments, is a TLR-9 targeted SNA. In some embodiments, the SNA comprises CpG oligonucleotides that are TLR9 agonists, and are referred to herein as CpG-SNA. In some embodiments, the TLR9 agonist is an oligonucleotide that comprises the nucleotide sequence of CpG7909 (5′-TCGTCGTTTTGTCGTTTTGTCGTT-3′ (SEQ ID NO: 1)). In some embodiments, the oligonucleotide comprises one or more phosphorothioate bonds. In some embodiment, all the bases in the oligonucleotide are linked via phosphorothioate bonds or the oligonucleotide has a phosphorothioate backbone.

In some embodiments, an oligonucleotide disclosed herein, such as a CpG oligonucleotide, comprises a spacer. As disclosed herein, when a CpG oligonucleotide comprises a spacer, the CpG oligonucleotide is attached to a spacer. In some embodiments, the CpG oligonucleotide is attached to the spacer through a covalent bond. In some embodiments, the spacer is oligoethylene glycol. In some embodiments, the oligoethylene glycol is a hexaethylene glycol (HEG). In some embodiments, the oligoethylene glycol is tetraethylene glycol (TEG). In some embodiments, the spacer does not comprise or consist of an oligonucleotide. In some embodiments, the spacer is an abasic spacer which does not include a nucleobase. In some embodiments, the spacer comprises a tetraethyleneglycol. In some embodiments, the oligonucleotide further comprises a hydrophobic group, such as a sterol. In some embodiments, the hydrophobic group is a cholesterol or a cholesteryl ester.

In a preferred embodiment, the oligonucleotide comprises or consists of:

(SEQ ID NO: 1)
5′-T*C*G*T*C*G*T*T*T*T*G*T*C*G*T*T*T*T*G*T*C*G*T*
T-3′/HEG/HEG/TEG Cholesteryl Ester/

The abbreviations used for this sequence are shown below:

| Abbreviation | Chemical Name |
|---|---|
| C | 2′-deoxy-P-cytidylyl |
| G | 2′-deoxy-P-guanylyl |
| T | 2′-deoxy-P-thymidylyl |
| HEG | Hexa(ethylene glycol)phosphodiester |
| TEG Cholesteryl Ester | (N-cholesteryl-3-aminopropyl)-triethyleneglycol- |
| | glyceryl-1-O-phosphodiester |
| * | Phosphorothioate internucleotide linkage |

In some embodiments, the oligonucleotide that comprises or consists of 5′-TCGTCGTTTTGTCGTTTTGTCGTT-3′(SEQ ID NO: 1). In some embodiments, the oligonucleotide that comprises or consists of 5′-T*C*G*T*C*G*T*T*T*T*G*T*C*G*T*T*T*T*G*T*C*G*T*T-3′/HEG/HEG/TEG Cholesteryl Ester/(SEQ ID NO: 1) is in a SNA, which is also referred to herein as CpG7909-SNA.

In some embodiments, a SNA disclosed herein, such as CpG7909-SNA, is formulated in DOPC and in a phosphate buffered saline (PBS) buffer that acts as a pH and osmolality modifier (solvent).

An oligonucleotide disclosed herein may be positioned on the exterior surface of the core. In some embodiments, at least or about 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 oligonucleotides or any range combination thereof are on the exterior surface of a liposome core or liposomal core.

In some embodiments, the SNA includes a neutral lipid. The neutral lipid may be, for example, 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE), any related phosphatidylcholine or neutral lipids available from commercial vendors.

In some embodiments, the checkpoint inhibitor is a molecule such as a monoclonal antibody, a humanized antibody, a fully human antibody, a fusion protein or a combination thereof or a small molecule. For instance, the checkpoint inhibitor inhibits a checkpoint protein which may be programmed cell death protein-1 (PD-1). In some embodiments, the checkpoint inhibitor inhibits or decreases the activity of a PD-1 or one or more of its ligands. The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows them to gain control of the PD-1 pathway and switch off T cells expressing PD-1 that may enter the tumor microenvironment, thus suppressing the anticancer immune response.

In some embodiments, the checkpoint inhibitor is an antibody that targets PD-1. In some embodiments, the antibody is pembrolizumab (formerly MK-3475 and lambrolizumab; KEYTRUDA®).

The checkpoint inhibitor may be a molecule such as a monoclonal antibody, a humanized antibody, a fully human antibody, a fusion protein or a combination thereof or a small molecule. For instance, the checkpoint inhibitor inhibits a checkpoint protein which may be CTLA-4, PD-L1, PDL2, PD-1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PD-L1, PDL2, PD-1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands.

In some embodiments, the anti-PD-1 antibody is BMS-936558 (nivolumab).

In other embodiments, the anti-CTLA-4 antibody is ipilimumab (trade name Yervoy, formerly known as MDX-010 and MDX-101).

In some embodiments, the anti-PD-1 antibody is REGN-2810 (cemiplimab).

In some embodiments, the anti-PD-1 antibody is PDR001 (spartalizumab).

In some embodiments, the checkpoint inhibitor is an anti-PD-L1 antibody.

In some embodiments, the anti-PD-L1 antibody is MPDL3280A (atezolizumab).

In some embodiments, the anti-PD-L1 antibody is MSB0010718C (avelumab).

In some embodiments, the anti-PD-L1 antibody is MEDI4736 (durvalumab).

In some embodiments, the method for treating cancer comprises administering a therapeutic dose of a spherical nucleic acid (SNA) comprising a CpG oligonucleotide linked through a spacer to an exterior surface of a liposome core having a diameter of less than about 40 nm and a checkpoint inhibitor, wherein the SNA is administered by intratumoral injection into multiple lesions at a dose of between about 16 mg and about 32 mg and the checkpoint inhibitor is administered by intravenous injection at a dose of between 180 and 220 mg, wherein the cancer is Merkel cell carcinoma or cutaneous squamous cell carcinoma.

In some embodiments, the SNA is administered at a dose (e.g., therapeutic dose) of or about 16 mg. In some embodiments, the SNA is administered at a dose of or about 32 mg. In some embodiments, the SNA is administered at a dose of or about 16 mg and the checkpoint inhibitor is administered at a dose of or about 200 mg. In some embodiments, the SNA is administered at a dose of or about 32 mg and the checkpoint inhibitor is administered at a dose of or about 200 mg. In some embodiments, the SNA is administered at a dose of or about 16 mg. In some embodiments, the SNA is administered at a dose of or about 32 mg. In some embodiments, the SNA is administered at a dose of or about 16 mg and the checkpoint inhibitor is administered at a dose of or about 350 mg. In some embodiments, the SNA is administered at a dose of or about 32 mg and the checkpoint inhibitor is administered at a dose of or about 350 mg. In some embodiments, the checkpoint inhibitor is an anti-PD-1 antibody. In some embodiments, the checkpoint inhibitor is pembrolizumab or cemiplimab. In some embodiments, the checkpoint inhibitor is an anti-PD-L1 antibody. In some embodiments, the checkpoint inhibitor is avelumab administered at a dose of or about 800 mg and the cancer is Merkel cell carcinoma.

As disclosed herein, the dose (e.g., a therapeutic dose) as it relates to administration of a SNA disclosed herein (e.g., without limitation the phrases “SNA is administered at a dose,” “SNA is administered to the subject at a fixed dose,” “total dose of a SNA disclosed herein and/or the total volume of a SNA disclosed herein is administered,” or “SNA dose administered”, etc.) refers to the total weight or total mass of active agent (i.e., total weight or total mass of the CpG oligonucleotides) that are part of the SNA and is being administered to the subject (e.g., subject with cancer).

In some embodiments, the SNA is administered at a dose (e.g., therapeutic dose) between 0.1 mg and 10 mg, between 0.2 mg and 10 mg, between 0.3 mg and 10 mg, between 0.4 mg and 10 mg, between 0.5 mg and 10 mg, between 0.6 mg and 10 mg, between 0.7 mg and 10 mg, between 0.8 mg and 10 mg, between 0.9 mg and 10 mg, between 1 mg and 10 mg, between 1 mg and 1000 mg, between 1 mg and 900 mg, between 1 mg and 800 mg, between 1 mg and 700 mg, between 1 mg and 600 mg, between 1 mg and 500 mg, between 1 mg and 450 mg, between 1 mg and 400 mg, between 1 mg and 350 mg, between 1 mg and 300 mg, between 1 mg and 250 mg, between 1 mg and 200 mg, between 1 mg and 150 mg, between 1 mg and 100 mg, between 1 mg and 90 mg, between 1 mg and 80 mg, between 1 mg and 70 mg, between 1 mg and 60 mg, between 1 mg and 60 mg, between 1 mg and 50 mg, between 1 mg and 49 mg, between 1 mg and 48 mg, between 1 mg and 47 mg, between 1 mg and 46 mg, between 1 mg and 45 mg, between 1 mg and 44 mg, between 1 mg and 43 mg, between 1 mg and 42 mg, between 1 mg and 41 mg, between 1 mg and 40 mg, between 1 mg and 39 mg, between 1 mg and 38 mg, between 1 mg and 37 mg, between 1 mg and 36 mg, between 1 mg and 35 mg, between 1 mg and 34 mg, between 1 mg and 33 mg, between 1 mg and 32 mg, between 1 mg and 31 mg, between 1 mg and 30 mg, between 1 mg and 29 mg, between 1 mg in 28 mg, between 1 mg and 27 mg, between 1 mg and 26 mg, between 1 mg and 25 mg, between 1 mg and 24 mg, between 1 mg and 23 mg, between 1 mg and 22 mg, between 1 mg and 21 mg, between 1 mg and 20 mg, between 1 mg and 19 mg, between 1 mg and 18 mg, between 1 mg and 17 mg, between 1 mg and 16 mg, between 1 mg and 15 mg, between 1 mg and 14 mg, between 1 mg and 13 mg, between 1 mg and 12 mg, between 1 mg and 11 mg, between 1 mg and 10 mg, between 1 mg and 9 mg, between 1 mg and 8 mg, between 1 mg and 7 mg, between 1 mg and 6 mg, between 1 mg and 5 mg, between 1 mg and 4 mg, between 1 mg and 2 mg, between 1 mg and 1.5 mg, between 1 mg and 3 mg, between 3 mg and 5 mg, between 5 mg and 7 mg, between 7 mg and 9 mg, between 9 mg and 14 mg, between 15 mg and 17 mg, between 18 mg and 31 mg, between 31 mg and 33 mg, between 0.5 mg and 2 mg, between 2 mg and 4 mg, between 11 mg and 13 mg, between 23 mg and 25 mg, between 2 mg and 31 mg, between 2 mg and 30 mg, between 2 mg and 29 mg, between 2 mg and 28 mg, between 2 mg and 27 mg, between 2 mg and 26 mg, between 2 mg and 25 mg, between 2 mg and 24 mg, between 2 mg and 23 mg, between 2 mg and 22 mg, between 2 mg and 21 mg, between 2 mg and 20 mg, between 2 mg and 19 mg, between 2 mg and 18 mg, between 2 mg and 17 mg, between 2 mg and 16 mg, between 2 mg and 15 mg, between 2 mg and 14 mg, between 2 mg and 13 mg, between 2 mg and 12 mg, between 2 mg and 11 mg, between 2 mg and 10 mg, between 2 mg and 9 mg, between 2 mg and 8 mg, between 2 mg and 7 mg, between 2 mg and 6 mg, between 2 mg and 5 mg, between 2 mg and 3 mg.

In some embodiments, the SNA is administered at a dose (e.g., therapeutic dose) of or about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 14 mg, 14.5 mg, 15 mg, 15.5 mg, 16 mg, 16.5 mg, 17 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, the SNA is administered at a dose (e.g., therapeutic dose) of at least or at least about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, the SNA is administered at a dose (e.g., therapeutic dose) greater than or greater than about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 40 mg, 14.5 mg, 50 mg, 15.5 mg, 60 mg, 16.5 mg, 70 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, the method for treating cancer comprises administering to a subject a spherical nucleic acid (SNA) and a checkpoint inhibitor, wherein the SNA comprises a core having an oligonucleotide shell comprised of CpG oligonucleotides positioned on the exterior of the core, wherein the SNA is administered to the subject at a fixed dose of between about 16 mg to about 32 mg to one solid tumor or tumor lesion or at a fixed dose of between about 16 mg to about 32 mg divided among two or more solid tumors or tumor lesions in the subject, wherein the SNA is administered within 24 hours of administration of the checkpoint inhibitor, to treat the cancer in the subject, wherein the cancer is Merkel cell carcinoma or cutaneous squamous cell carcinoma.

As disclosed herein, the terms “solid tumor”, “tumor lesion” or “target lesion” are used interchangeably. In some embodiments, a solid tumor, tumor lesion or target lesion is an accessible solid tumor, accessible tumor lesion or an accessible target lesion. In some embodiments, the total dose of a SNA disclosed herein and/or the total volume of a SNA disclosed herein is administered to one solid tumor or tumor lesion in a subject in methods for treating cancer. In some embodiments, the subject has more than one solid tumor or tumor lesion. If the subject has more than one solid tumor or tumor lesion, the total dose of a SNA disclosed herein and/or the total volume of a SNA disclosed herein is divided equally among the solid tumors or tumor lesions in the subject. In some embodiments, the total dose of SNA and/or total volume of SNA administered to each solid tumor or tumor lesion in the subject may depend on various factors, such as on the number of injectable and/or accessible solid tumors or tumor lesions in the subject, the size of the smallest injectable and/or accessible solid tumor or tumor lesion in the subject, the concentration of SNA in a solution or formulation, and/or a minimum concentration for which syringe stability data are available.

For instance, in a non-limiting example, if a SNA disclosed herein is available in a solution or formulation at a concentration of 20 mg/mL, the subject is to receive a total dose of SNA of 4 mg and the subject has four solid tumors or tumor lesions, then each solid tumor or tumor lesion is to be injected with 0.050 mL of the SNA solution or formulation (at the concentration of 20 mg/mL). In a further non-limiting example, if the subject is to receive a total dose of the SNA of 4 mg and the subject has three solid tumors or tumor lesions, then each solid tumor or tumor lesion is to be injected with 0.067 mL of SNA solution or formulation (at the concentration of 20 mg/mL). In these non-limiting examples and for clarity, the concentration 20 mg/mL refers to the 20 mg of CpG oligonucleotides per mL of solution or formulation.

Thus, the total volume of SNA solution or formulation for a subject and the volume of SNA for each solid tumor or tumor lesion in the subject depends on the concentration of SNA solution or formulation, the dose of SNA for the subject and the number of injectable and/or accessible solid tumors or tumor lesions in the subject.

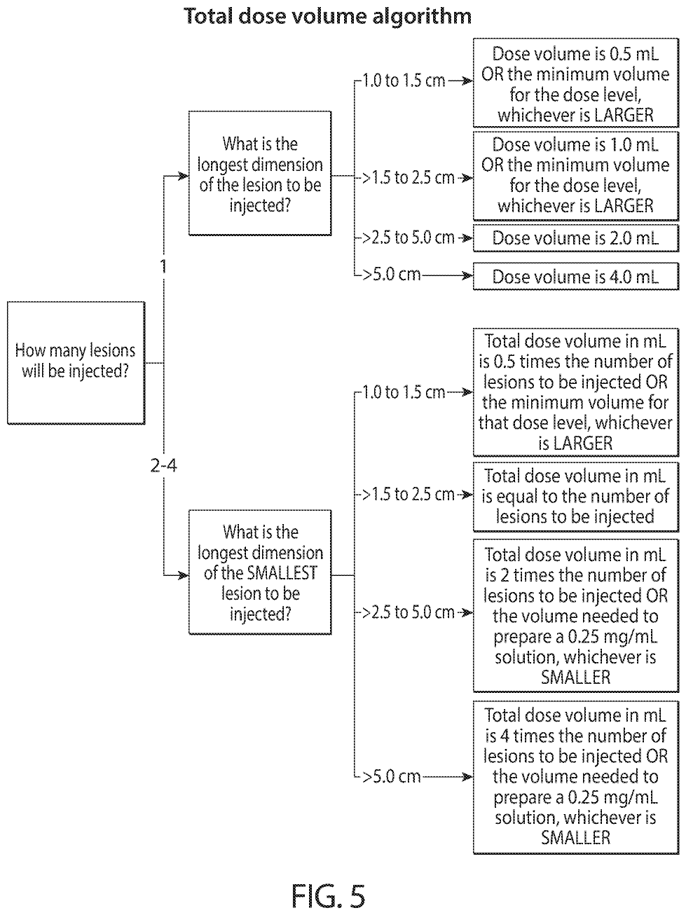

In some embodiments, the injectable and/or accessible solid tumors or tumor lesions are of different sizes. If the solid tumors or tumor lesions are of different sizes, the volume of SNA solution or formulation for each solid tumor or tumor lesion in the subject will depend on the longest dimension of the smallest lesion to be injected as described, for instance as a non-limiting example, in FIG. 5. In some embodiments, the total dose of SNA and/or the total volume of SNA is delivered or administered to one accessible tumor lesion or solid tumor, or divided among two accessible tumor lesions or solid tumors, three accessible tumor lesions or solid tumors, four accessible tumor lesions or solid tumors, five accessible tumor lesions or solid tumors, six accessible tumor lesions or solid tumors, seven accessible tumor lesions or solid tumors, eight accessible tumor lesions or solid tumors, nine accessible tumor lesions or solid tumors, or ten or more than 10 accessible tumor lesions or solid tumors. In some embodiments, the total dose of SNA and/or the total volume of SNA is delivered or administered into no more than four accessible tumor lesions or solid tumors. In some embodiments, the SNA is administered intratumorally (IT), cutaneously, subcutaneously or into a lymph node.

In some embodiments, the total dose of SNA and/or the total volume of SNA is not divided equally among the solid tumors or tumor lesions in a subject that has more than one solid tumor or tumor lesion. In some embodiments, the SNA dose administered to one solid tumor or tumor lesion is greater than the SNA dose that would be administered to one solid tumor or tumor lesion if the SNA dose was divided equally among all the accessible and/or injectable solid tumors or tumor lesions in the subject. In some embodiments, administration of a SNA or administration of a SNA in combination with a checkpoint inhibitor to a subject with cancer results in reduced diameter of one or more solid tumors or tumor lesions or complete disappearance of one or more solid tumors or tumor lesions which have not directly received a dose of the SNA (e.g., the SNA has not been injected into the solid tumor or tumor lesion).

For instance, in a non-limiting example, a subject having three solid tumors or tumor lesions is administered a total SNA dose of 4 mg, wherein one solid tumor or tumor lesion receives a SNA dose of 2 mg and each of the two remaining solid tumors or tumor lesions receive a SNA dose of 1 mg, for a total SNA dose of 4 mg administered to the subject. In some embodiments, the SNA dose administered to one solid tumor or tumor lesion is less than the SNA dose that would be administered to one solid tumor or tumor lesion if the SNA dose was divided equally among all the accessible and/or injectable solid tumors or tumor lesions in the subject. For instance, in a non-limiting example, a subject having three solid tumors or tumor lesions is administered a total SNA dose of 4 mg, wherein one solid tumor or tumor lesion receives a SNA dose of 0.5 mg and each of the two remaining solid tumors or tumor lesions receive a SNA dose of 1.75 mg, for a total SNA dose of 4 mg administered to the subject.

In some embodiments, the term “multiple lesions” refers to two or more solid tumors, tumor lesions or target lesions. In some embodiments, the term multiple lesions refers to at least two, at least three, at least four, at least five, at least six, at least seven, at least eight, at least nine, at least 10, at least 11, or at least 12 solid tumors, tumor lesions or target lesions, or any range or combination thereof.

In some embodiments, a dose is a therapeutic dose. In some embodiments, the SNA is administered at a dose (e.g., therapeutic dose) that results in a measurable concentration of SNA in the plasma of a subject. In some embodiments, the SNA is administered at a dose (e.g., therapeutic dose) that results in a measurable concentration of SNA in the plasma of a subject of between 0.1 ng/mL and 1000 ng/mL. In some embodiments, the SNA is administered at a dose (e.g., therapeutic dose) that results in a measurable concentration of SNA in the plasma of a subject of between 0.2 ng/mL and 1000 ng/mL, between 1 ng/mL and 1000 ng/mL, between 2 ng/mL and 1000 ng/mL, between 3 ng/mL and 1000 ng/mL, between 4 ng/mL and 1000 ng/mL, between 5 ng/mL and 1000 ng/mL, between 6 ng/mL and 1000 ng/mL, between 7 ng/mL and 1000 ng/mL, between 8 ng/mL and 1000 ng/mL, between 9 ng/mL and 1000 ng/mL, or between 10 ng/mL and 1000 ng/mL.

In some embodiments, the SNA is administered at a dose (e.g., therapeutic dose) that results in concentrations of SNA in the plasma of a subject of between 1 ng/mL and 200 ng/mL, between 1 ng/mL and 100 ng/mL, between 1 ng/mL and 90 ng/mL, between 1 ng/mL and 80 ng/mL, between 1 ng/mL and 70 ng/mL, between 1 ng/mL and 60 ng/mL, between 1 ng/mL and 50 ng/mL, between 1 ng/mL and 40 ng/mL, between 1 ng/mL and 30 ng/mL, between 1 ng/mL and 20 ng/mL, between 1 ng/mL and 10 ng/mL, between 1 ng/mL and 9 ng/mL, between 1 ng/mL and 8 ng/mL, between 1 ng/mL and 7 ng/mL, between 1 ng/mL and 6 ng/mL, between 1 ng/mL and 5 ng/mL, between 1 ng/mL and 4 ng/mL, between 1 ng/mL and 3 ng/mL, or between 1 ng/mL and 2 ng/mL.

In some embodiments, the checkpoint inhibitor is administered at a dose (e.g., therapeutic dose) of between 50 mg and 1000 mg, between 50 mg and 750 mg, between 50 mg and 500 mg, between 50 mg and 400 mg, between 50 mg and 350 mg, between 50 mg and 300 mg, between 50 mg and 290 mg, between 50 mg and 280 mg, between 50 mg and 270 mg, between 50 mg and 260 mg, between 50 mg and 250 mg, between 50 mg and 240 mg, between 50 mg and 230 mg, between 50 mg and 220 mg, between 50 mg and 210 mg, between 50 mg and 200 mg, between 60 mg and 200 mg, between 70 mg and 200 mg, between 80 mg and 200 mg, between 90 mg and 200 mg, between 100 mg and 200 mg, between 110 mg and 200 mg, between 120 mg and 200 mg, between 130 mg and 200 mg, between 140 mg and 200 mg, between 150 mg and 200 mg, between 160 mg and 200 mg, between 170 mg and 200 mg, between 180 mg and 200 mg, or between 190 mg and 200 mg, between 200 mg and 500 mg, between 200 mg and 450 mg, between 200 mg and 440 mg, between 200 mg and 430 mg, between 200 mg and 420 mg, between 200 mg and 410 mg, between 200 mg and 400 mg, between 200 mg and 390 mg, between 200 mg and 380 mg, 200 mg and 370 mg, between 200 mg and 360 mg, between 200 mg and 350 mg, between 210 mg and 350 mg, between 220 mg and 350 mg, between 230 mg and 350 mg, between 240 mg and 350 mg, between 250 mg and 350 mg, between 260 mg and 350 mg, between 270 mg and 350 mg, between 280 mg and 350 mg, between 290 mg and 350 mg, between 300 mg and 350 mg, between 310 mg and 350 mg, between 320 mg and 350 mg, between 330 mg and 350 mg, or between 340 mg and 350 mg.

In some embodiments, the checkpoint inhibitor is administered at a dose (e.g., therapeutic dose) of or about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg, or any range or combination thereof. In some embodiments, the checkpoint inhibitor is pembrolizumab administered at a dose of between 180 mg and 220 mg, avelumab administered at a dose of between 700 mg and 900 mg, or cemiplimab administered at a dose of between 330 mg and 370 mg.

In some embodiments, the checkpoint inhibitor is administered at a dose (e.g., therapeutic dose) of at least or at least about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg, or any range or combination thereof.

In some embodiments, the checkpoint inhibitor is administered at a dose (e.g., therapeutic dose) greater than or greater than about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg, or any range or combination thereof.

In some embodiments, the checkpoint inhibitor is administered at a dose (e.g., therapeutic dose) of or about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, or 50 mg/kg, or any range or combination thereof.

In some embodiments, a SNA disclosed herein is administered at a dose (e.g., therapeutic dose) disclosed herein in combination with a checkpoint inhibitor disclosed herein at a dose (e.g., a therapeutic dose) disclosed herein in methods for treating cancer. For instance, a non-limiting example includes the administration of a SNA disclosed herein to a subject at a dose of 2 mg and the administration of a checkpoint inhibitor disclosed herein at a dose of 200 mg. The combination of any of the dose amounts or dose ranges disclosed herein are also contemplated herein in methods for treating cancer in a subject.

In some embodiments, a dose disclosed herein is considered a fixed dose or a discrete dose. In some embodiments, a dose disclosed herein can be adjusted to depend on body weight or made dependent on body weight. A non-limiting example includes a dose of 2 mg of SNA, as disclosed herein, that can also be administered as 2 mg/kg/body weight, which depends on kg/body weight.

In some embodiments, the cancer is melanoma, mucosal melanoma, cutaneous melanoma, metastatic malignant melanoma, renal cancer, clear cell carcinoma, prostate cancer, hormone refractory prostate adenocarcinoma, breast cancer, colon cancer, lung cancer, non-small cell lung cancer, bone cancer, pancreatic cancer, pancreatic adenocarcinoma, skin cancer, cancer of the head or neck, cutaneous or intraocular malignant melanoma, uterine cancer, ovarian cancer, rectal cancer, stomach cancer, testicular cancer, thyroid cancer, anaplastic thyroid cancer, uterine cancer, carcinoma of the fallopian tubes, carcinoma of the endometrium, carcinoma of the cervix, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, biliary tract cancer, brain cancer, breast cancer, cervical cancer, choriocarcinoma, esophageal cancer, gastric cancer, an intraepithelial neoplasm, lymphoma, liver cancer, neuroblastoma, oral cancer, sarcomas, hairy cell leukemia, chronic myelogenous leukemia, cutaneous T-cell leukemia, multiple myeloma, renal cell carcinoma, lymphoma, bladder cancer, non-small cell lung cancer (NSCLC), glioblastoma multiforme, Merkel cell carcinoma, cutaneous squamous cell carcinoma, melanoma or squamous cell carcinoma of the head and neck, environmentally induced cancers including those induced by asbestos, or any combinations thereof. In some embodiments, the cancer is not melanoma.

In some embodiments, the cancer is a sarcoma, including pleomorphic sarcoma, gastrointestinal stromal tumor (GIST), liposarcoma, leiomyosarcoma, synovial sarcoma, malignant peripheral nerve sheath tumor, rhabdomyosarcoma, angiosarcoma, fibrosarcoma, dermatofibrosarcoma protuberans, epithelioid sarcoma, myxoma, mesenchymoma, vascular sarcoma, neurilemmoma, bone sarcoma, osteosarcoma, Ewing's sarcoma, chondrosarcoma, Kaposi sarcoma, solitary fibrous tumor, chordoma, desmoid-type fibromatosis, fibroblastic sarcoma, giant cell tumor of the bone, gynaecological sarcoma, soft tissue sarcoma, angioleiomyoma, leiomyoma, smooth muscle sarcoma, fibrohistiocytic sarcoma.

In some embodiments, the cancer is a rare cancer such as dermatofibroma protuberans, angiosarcoma of the skin, non-HIV-related Kaposi's sarcoma, sebaceous cell carcinoma, and eccrine carcinoma. In some embodiments, the cancer is an immunogenic cancer or a cancer associated with or that arises from a viral infection. In some embodiments, the viral infection is caused by the Merkel cell polyoma virus. Without wishing to be bound by theory, the Merkel cell polyoma virus may be associated, at least in some instances, with Merkel cell carcinoma. In some embodiments, the viral infection is caused by the Epstein-Barr virus (e.g., associated with Burkitt's lymphoma), the human papilloma virus (e.g., associated with cervical cancer), Hepatitis B virus or Hepatitis C virus (e.g., possibly associated with some forms of hepatocellular carcinoma).

In some embodiments, the cancer is characterized as microsatellite instability high, or MSI-H, or mismatch repair deficient, or dMMR. MSI-H or dMMR cancers are characterized by defects in DNA replication, particularly in the microsatellite regions. The presence of MSI-H and dMMR tumors has been reported in diverse cancer types, including colon, colorectal, endometrial, biliary, gastric, gastroesophageal junction, pancreatic, small intestinal, breast, triple negative breast, prostate, bladder, esophageal, sarcoma, thyroid, retroperitoneal adenocarcinoma, small cell lung, ovarian, pancreatic, prostate, central nervous system, and non-small cell lung cancers.

In some embodiments, the cancer is a metastatic cancer that expresses programmed death-ligand 1 (PD-L1) or PD-1.

In some embodiments, the cancer is refractory or resistant to treatment with a checkpoint inhibitor antibody, such as a PD-1 antibody.

In some embodiments, a method for treating cancer comprises administering a SNA and a checkpoint inhibitor disclosed herein to a subject that has a solid tumor. A solid tumor as used herein refers to an abnormal mass of tissue that usually does not contain cysts or liquid areas. Solid tumors may be benign (not cancer), or malignant (cancer). Different types of solid tumors are named for the type of cells that form them. Non-limiting examples of solid tumors include sarcomas, carcinomas, and lymphomas. Other solid tumors arising from other tissues, organs or areas of the body are also contemplated herein. In some embodiments, the solid tumor or tumor lesion is on or near the skin, on or near soft tissue and in or near a lymph node. In some embodiments, the solid tumor or tumor lesion is on or near cutaneous soft tissue or subcutaneous soft tissue.

In some embodiments, the method for treating cancer further comprises antigen. In some embodiments, the antigen is a cancer antigen. In some embodiments, the antigen is positioned on the surface of a SNA disclosed herein. In some embodiments, the antigen is encapsulated in the core of a SNA disclosed herein. In some embodiments, the antigen, the SNA and/or checkpoint inhibitor are administered in the same formulation. In some embodiments, the antigen, the SNA and/or checkpoint inhibitor are in administered in separate formulations. In some embodiments, the antigen and SNA are administered in the same formulation and the checkpoint inhibitor is in a separate formulation.

In some embodiments, a SNA disclosed herein is administered in conjunction or in combination with a checkpoint inhibitor disclosed herein. The terms “in conjunction with,” “in combination with,” or “co-administered” refers to a therapy which involves the delivery of the two therapeutics, such as a SNA and a checkpoint inhibitor, to a subject. The two therapeutics may be administered together in a single composition, at the same time, in separate compositions using the same or different routes of administration, or at different times using the same or different routes of administration. In a preferred embodiment, the two therapeutics, such as a SNA disclosed herein and a checkpoint inhibitor disclosed herein, are administered in separate compositions using different routes of administration at the same time or substantially the same time. In some embodiments, a SNA disclosed herein and a checkpoint inhibitor disclosed herein are administered within five days, within four days, within 72 hours, within 48 hours, within 24 hours, within 12 hours, within 6 hours, within 4 hours, within 3 hours, within 2 hours, within 1 hour, within 30 minutes, within 10 minutes, within 5 minutes, within 1 minute of administration of each other. In some embodiments, the SNA and the checkpoint inhibitor are administered substantially simultaneously or substantially at the same time (e.g., during the time the subject is receiving the checkpoint inhibitor).

In some embodiments, the method for treating cancer comprises administering to a subject a spherical nucleic acid (SNA) and a checkpoint inhibitor, wherein the SNA comprises a core having an oligonucleotide shell comprised of CpG oligonucleotides positioned on the exterior of the core, wherein the SNA is administered at a dose of between about 16 mg to about 32 mg every three weeks, wherein the checkpoint inhibitor is administered at a dose of between 180 and 220 mg every three weeks, wherein the SNA is administered within 24 hours of the administration of the checkpoint inhibitor, and wherein the SNA and the checkpoint inhibitor are administered through different routes of administration to treat the cancer in the subject, wherein the cancer is Merkel cell carcinoma or cutaneous squamous cell carcinoma.

In some embodiments, a SNA or checkpoint inhibitor disclosed herein is administered once a day, once every three days, once a week, once every two weeks, once every three weeks, once every four weeks, once every five weeks, once every six weeks, once every seven weeks, once every eight weeks, once every nice weeks, once every 10 weeks, once every 12 weeks, once every 18 weeks, once every 24 weeks, once a month, once every two months, once every three months, once every four months, once every five months, once every six months, once every seven months, once every eight months, once every nine months, once every 10 months, once every 11 months, once a year, once every two years, once every three years, once every four years. In some embodiments, the checkpoint inhibitor is pembrolizumab administered every three weeks, avelumab administered every two weeks, or cemiplimab administered every three weeks.

In some embodiments, a SNA disclosed herein is administered once a week, twice a week or three times per week, for four weeks, six weeks, eight weeks, 10 weeks, 12 weeks, 14 weeks, 16 weeks, 18 weeks, 20 weeks, 24 weeks, one month, two months, three months, four months, five months, six months, seven months, eight months, nine months, 10 months, 11 months, one year, two years, three years, four years, five years, six years. In some embodiments and SNA disclosed herein is administered every three weeks for four weeks, six weeks, eight weeks, 10 weeks, 12 weeks, 14 weeks, 16 weeks, 18 weeks, 20 weeks, 24 weeks, one month, two months, three months, four months, five months, six months, seven months, eight months, nine months, 10 months, 11 months, one year, two years, three years, four years, five years, six years, seven years, eight years, nine years, 10 years.

In some embodiments, the duration of the method for treating cancer with a SNA disclosed herein and a checkpoint inhibitor disclosed herein is for 3 months, for six months, for nine months, for one year, for 1.5 years, for two years, for 2.5 years, for 3 years, for 3.5 years, for 4 years, for 4.5 years, for 5 years, for 5.5 years, for 6 years, for 6.5 years, for 7 years, for 7.5 years, for 8 years, for 8.5 years, for 9 years, for 9.5 years, for 10 years, for 15 years, for 20 years or more than 20 years.

In a preferred embodiment, the SNA is administered every three weeks. In some embodiments, the SNA is administered about or at least about every four weeks, five weeks, six weeks, 2 months, three months, six months, nine months, one year, 1.5 years, two years, 2.5 years, three years, 3.5 years, four years, 4.5 years, five years, 5.5 years, or six years. In a preferred embodiment, the checkpoint inhibitor disclosed herein is administered every three weeks for or at least about three months, six months, nine months, one year, 1.5 years, two years, 2.5 years, three years, 3.5 years, four years, 4.5 years, five years, 5.5 years, or six years.

In some embodiments, the first dose of the SNA disclosed herein is administered one day, two days, three days, four days, five days, six days, seven days, eight days, nine days, 10 days, 11 days, 12 days, 30 days, 14 days, 15 days, 16 days, one week, 1.5 weeks, two weeks, 2.5 weeks, three weeks, 3.5 weeks, four weeks, 4.5 weeks, five weeks, 5.5 weeks, six weeks, 6.5 weeks, seven weeks, 7.5 weeks, eight weeks, 8.5 weeks, nine weeks, 9.5 weeks, the weeks, 10.5 weeks, 11 weeks, 11.5 weeks, 12 weeks, three months, four months, five months, six months, seven months, eight months, nine months, 10 months, 11 months, or one year, 1.5 years, two years, 2.5 years, three years, 3.5 years, four years, 4.5 years, five years, 5.5 years, or six years, after the first dose of a checkpoint inhibitor disclosed herein is administered to the subject.

In a preferred embodiment, a checkpoint inhibitor disclosed herein and the SNA disclosed herein are administered every three weeks for at least or about two years.

In some embodiments, a SNA disclosed herein is administered to a subject with a cancer that is refractory, resistant or non-responsive to therapy with a checkpoint inhibitor, such as an inhibitor of PD-1 (e.g., a PD-1 antibody) or an inhibitor of PD-L1 (e.g., a PD-L1 antibody), or both an inhibitor of PD-1 and an inhibitor of PD-L1. In some embodiments, a SNA disclosed herein is administered to a subject with a cancer that is refractory, resistant or non-responsive to therapy with avelumab, pembrolizumab or cemiplimab. In some embodiments, a SNA disclosed herein, alone or in combination with a checkpoint inhibitor disclosed herein, is administered to a subject with metastatic Merkel cell carcinoma, or metastatic cutaneous squamous cell carcinoma or locally advanced cutaneous squamous cell carcinoma who is not a candidate for curative surgery or curative radiation.

In some embodiments, a SNA disclosed herein, alone or in combination with a checkpoint inhibitor disclosed herein, is administered to a subject with a cancer that is progressive (or shows the characteristic(s) of Progressive Disease (PD), as measured by the RECIST criteria and disclosed herein). PD is defined by the RECIST criteria as having at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression). In some embodiments, a SNA disclosed herein, alone or in combination with a checkpoint inhibitor disclosed herein, is administered to a subject with a cancer that is stable (or shows the characteristic(s) of Stable Disease (SD) as measured by the RECIST criteria and disclosed herein). SD is defined in the RECIST criteria as either sufficient shrinkage to qualify for partial response, nor sufficient increase to qualify for PD, taking as reference the smallest sum diameters while on study.

In some embodiments, administration of a SNA disclosed herein, alone or in combination with a checkpoint inhibitor disclosed herein, to a subject with a cancer (e.g., melanoma, Merkel cell carcinoma, cutaneous squamous cell carcinoma, head and neck squamous cell carcinoma, mucosal melanoma) that is progressive or shows one or characteristics of PD causes the cancer to become stable or show characteristics of stable disease.

In some embodiments, the cancer is stable or stable disease for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, at least 40 weeks, at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, at least 40 months, at least one year, at least two years, at least three years, at least four years, at least five years, at least six years, at least seven years, at least eight years, at least nine years, at least 10 years, at least 11 years or at least 12 years in the subject.

In some embodiments, provided herein are methods of treatment involving administering a combination therapy of an SNA and a checkpoint inhibitor to an identified subject or patient having a tumor, where the subject or patient is identified by at least one of: (a) having an intact functioning immune system; (b) identifying the presence of a threshold amount of PD-L1 expression in the tumor (i.e. greater than or equal to 50% of cells being PD-L1 positive); (c) identifying the presence of a threshold amount of one or more of a T-effector associated gene and/or one or more of an interferon-y associated gene (e.g., level of gene expression as measured in the subject by methods known to one of ordinary skill in the art is higher than a pre-determined value); and (d) meeting a threshold level of tumor mutational burden (TMB).

In some embodiments, a cancer is characterized or analyzed using tumor tissue (e.g., a biopsy sample) obtained from a cancer patient or subject with cancer, image(s) of tissue sections (e.g., immunohistochemical staining), a blood sample, and gene expression. These methods can identify which subjects or patients have the greatest potential to respond to immune-mediated cancer therapy (e.g., atezolizumab, pembrolizumab or ipilmumab) or can be indicative of desired immune activation in a subject with cancer.

For instance, a certain level of one or more of cytokine expression, chemokine expression and immune cell activation measured in a blood sample or in a biopsy sample from a subject with cancer (e.g., melanoma, Merkel cell carcinoma, cutaneous squamous cell carcinoma, head and neck squamous cell carcinoma, mucosal melanoma) after administration of a SNA disclosed, a checkpoint inhibitor disclosed, or of both a SNA disclosed and checkpoint inhibitor disclosed, can be indicative of desired immune activation in the subject with cancer.

In some embodiments, the administration of the SNA alone at a dose (e.g., a therapeutic dose, such as 2 mg, 4 mg, 8 mg, 16 mg or 32 mg) or administration of the SNA at a dose (e.g., a therapeutic dose, such as 2 mg, 4 mg, 8 mg, 16 mg or 32 mg) in combination with a checkpoint inhibitor at a dose (e.g., therapeutic dose, such as 200 mg, 350 mg or 800 mg) results in one or more of increased cytokine expression, increased chemokine expression, or increased immune cell activation (e.g., CD4⁺ T cells, CD8⁺ T cells, monocytes, natural killer cells and lymphocytes) by at least or about 5%, at least or about 10%, at least or about 15%, at least or about 16%, at least or about 17%, at least or about 18%, at least or about 19%, at least or about 20%, at least or about 21%, at least or about 22%, at least or about 23%, at least or about 24%, at least or about 25%, at least or about 26%, at least or about 27%, at least or about 28%, at least or about 29%, at least or about 30%, at least or about 31%, at least or about 32%, at least or about 33%, at least or about 34%, at least or about 35%, at least or about 36%, at least or about 37%, at least or about 38%, at least or about 39%, at least or about 40%, at least or about 41%, at least or about 42%, at least or about 43%, at least or about 44%, at least or about 45%, at least or about 46%, at least or about 47%, at least or about 48%, at least or about 49%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, relative to a reference level.

In some embodiments, administration of a SNA disclosed herein alone at a dose (e.g., a therapeutic dose, such as 2 mg, 4 mg, 8 mg, 16 mg or 32 mg) or administration of a SNA disclosed herein at a dose (e.g., a therapeutic dose, such as 2 mg, 4 mg, or 8 mg, 16 mg or 32 mg) in combination with a checkpoint inhibitor at a dose (e.g., therapeutic dose, such as 200 mg, 350 mg, or 800 mg) results in one or more of increased cytokine expression, increased chemokine expression, or increased immune cell activation (e.g., CD4⁺ T cells, CD8⁺ T cells, monocytes, natural killer cells, and lymphocytes) by at least or about 100%, at least or about 150%, at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 11-fold, at least or about 12-fold, at least or about 13-fold, at least or about 14-fold, at least or about 15-fold, at least or about 16-fold, at least or about 17-fold, at least or about 18-fold, at least or about 19-fold, at least or about 20-fold, at least or about 21-fold, at least or about 22-fold, at least or about 23-fold, at least or about 24-fold, at least or about 25-fold, at least or about 26-fold, at least or about 27-fold, at least or about 28-fold, at least or about 29-fold, at least or about 30-fold, at least or about 40-fold, at least or about 50-fold, at least or about 60-fold, at least or about 70-fold, at least or about 80-fold, at least or about 90-fold, at least or about 100-fold or more, relative to a reference level. In some embodiments, administration of a SNA disclosed herein alone at a dose (e.g., a therapeutic dose, such as 2 mg, 4 mg, 8 mg, 16 mg or 32 mg) or administration of a SNA disclosed herein at a dose (e.g., a therapeutic dose, such as 2 mg, 4 mg, or 8 mg, 16 mg or 32 mg) in combination with a checkpoint inhibitor at a dose (e.g., therapeutic dose, such as 200 mg) results in one or more of increased cytokine expression, increased chemokine expression, or increased immune cell activation (e.g., CD4⁺ T cells, CD8⁺ T cells, monocytes, natural killer cells, and lymphocytes) at the solid tumor or tumor lesion that was intratumorally injected or both at the solid tumor or tumor lesion that was intratumorally injected and a witness solid tumor or tumor lesion that was not intratumorally injected in the subject.

In some embodiments, the cytokine or chemokine is interferon (IFN)-α, IFN-γ, interleukin (IL)-10, IL-12p40, IL-1(3, IL-1RA, IL-2, IL-6, IL-8, interferon gamma-induced protein (IP)-10, monocyte chemoattractant protein (MCP)-1, or tumor necrosis factor alpha (TNF)-α. In some embodiments, the immune cell is a lymphocyte. In some embodiments, administration of a SNA disclosed herein alone at a dose (e.g., a therapeutic dose, such as a therapeutic dose of 2 mg, 4 mg, 8 mg, 16 mg or 32 mg of SNA) or administration of a SNA disclosed herein at a dose (e.g., a therapeutic dose, such as a therapeutic dose of 2 mg, 4 mg, or 8 mg of SNA) in combination with a checkpoint inhibitor at a dose (e.g., therapeutic dose, such as 200 mg, 350 mg, or 800 mg) results in dose-dependent lymphocyte activation. In some embodiments, administration of a SNA disclosed herein alone at a dose (e.g., a therapeutic dose, such as a therapeutic dose of 2 mg, 4 mg, 8 mg, 16 mg or 32 mg of SNA) or administration of a SNA disclosed herein at a dose (e.g., a therapeutic dose, such as a therapeutic dose of 2 mg, 4 mg, or 8 mg of SNA) in combination with a checkpoint inhibitor at a dose (e.g., therapeutic dose, such as 200 mg) results in dose-dependent IL-12p40, IL-1RA, IP-10, and MCP-1 induction.

In some embodiments, the immune cell is a B-lymphocyte (e.g., All: CD3−/CD19+/CD45+; Activated B cells: CD3−/CD19+/CD45+/CD86+), a T-lymphocyte (e.g., All: CD3+/CD45+; Activated T cells: CD3+/CD45+/CD69+), a natural killer (NK) cell (e.g., All: CD3−/CD16+/CD19−/CD45+/CD56+; Activated NK cell: CD3−/CD16+/CD19−/ CD45+/CD56+/CD69+), a monocyte (e.g., All: CD14+/CD45+; Activated monocytes: CD14+/CD45+/CD86+; CD14+/CD45+/CD169+), a plasmacytoid dendritic cell (e.g., All: CD14−/CD11c−/CD16−/CD19—/CD45+/CD56−/CD123+/HLA-DR+; Activated pDCs: CD11c−/CD14−/CD16−/CD19−/CD45+/CD56−/CD86+/CD123+/HLA-DR+), a cytotoxic T cell, or a T helper cell.

In some embodiments, the threshold amount of PD-L1 expression in the tumor is when greater than or equal to 55% of cells are PD-L1 positive, greater than or equal to 60% of cells are PD-L1 positive, greater than or equal to 65% of cells are PD-L1 positive, greater than or equal to 70% of cells are PD-L1 positive, greater than or equal to 75% of cells are PD-L1 positive, greater than or equal to 80% of cells are PD-L1 positive, greater than or equal to 85% of cells are PD-L1 positive, greater than or equal to 90% of cells are PD-L1 positive, greater than or equal to 95% of cells are PD-L1 positive, or greater than or equal to 99% of cells are PD-L1 positive. In some embodiments, the cell that is PD-L1 positive is a tumor cell. In some embodiments, the cell that is PD-L1 positive is a tumor-infiltrating immune cell. In some embodiments, the cell that is a PD-L1 positive is a tumor-infiltrating immune cell and a tumor cell.

In some embodiments, the subject is a mammal. In some embodiments, the subject is a primate. In some embodiments, the subject is a human. In some embodiments, the mammal is a vertebrate animal including, but not limited to, a mouse, rat, dog, cat, horse, cow, pig, sheep, goat, turkey, chicken, monkey, fish (e.g., aquaculture species, salmon, etc.). Thus, the disclosure herein can also be used to treat diseases or disorders, such as cancer, in human or non-human subjects.

For use in therapy, a therapeutic dose of a SNA or checkpoint inhibitor disclosed herein is administered to a subject by any mode that delivers the SNA and/or checkpoint inhibitor to the desired surface (e.g., intratumoral, cutaneous, subcutaneous, nodal, systemic, etc.). The SNA and/or checkpoint can be administered in a pharmaceutical composition that is prepared by any means known one of ordinary skill in the art. Routes of administration include but are not limited to oral, parenteral, intramuscular, intranasal, sublingual, intratracheal, inhalation, ocular, vaginal, and rectal. In some embodiments, preferred routes of administration of a SNA or checkpoint inhibitor disclosed herein include intravenous (IV) injection, IV infusion, intratumoral injection, cutaneous injection, nodal injection and subcutaneous injection. In a preferred embodiment, the SNA is administered through cutaneous, subcutaneous, or intratumoral injections and the checkpoint inhibitor is administered through IV infusion.

In some embodiments, a SNA and/or checkpoint inhibitor disclosed herein is administered to a deep visceral lesion (e.g., liver lesion or lung metastases). In some embodiments, the administration to a deep visceral lesion requires radiological control via computed tomography (CT) or magnetic resonance imaging (MRI). Other methods of radiological control known to one of ordinary skill in the art are also contemplated herein. In some embodiments, the administration to a deep visceral lesion requires ultrasound-guided or endoscope-guided injection and delivery. In some embodiments, the deep visceral lesion is in an internal organ of the body of the subject. In some embodiments, the deep visceral lesion is in the liver, heart, pancreas, kidney, stomach, lung, or intestines.

In some embodiments, the checkpoint inhibitor is administered by IV infusion for an amount of time that is between 5 minutes and 12 hours. In some embodiments, the checkpoint inhibitor is administered by IV infusion for or about 5 minutes, 10 minutes, 15 minutes, 20 minutes, 31 minutes, 22 minutes, 23 minutes, 24 minutes, 25 minutes, 26 minutes, 27 minutes, 28 minutes, 29 minutes, 30 minutes, 31 minutes, 32 minutes, 33 minutes, for 34 minutes, 35 minutes, 36 minutes, 37 minutes, 38 minutes, 39 minutes, 40 minutes, 45 minutes, 50 minutes, 55 minutes, 60 minutes, 1.5 hours, two hours, 2.5 hours, three hours, 3.5 hours, four hours, 4.5 hours, five hours, 5.5 hours, six hours, eight hours, 10 hours, or 12 hours. In some embodiments, the checkpoint inhibitor is pembrolizumab administered by IV infusion for 30 minutes, avelumab administered by IV infusion for 60 minutes, or cemiplimab administered by IV infusion for 30 minutes. In some embodiments, the checkpoint inhibitor is administered until disease progression or unacceptable toxicity.

In some embodiments, the checkpoint inhibitor is in a solution at a concentration of between 0.1 mg/mL and 50 mg/mL. In some embodiments, the checkpoint inhibitor is in a solution at a concentration of between 1 mg/mL and 50 mg/mL, between 5 mg/mL and 50 mg/mL, between 10 mg/mL and 50 mg/mL, between 20 mg/mL and 50 mg/mL, between 30 mg/mL and 50 mg/mL, or between 40 mg/mL and 50 mg/mL. In some embodiments, the checkpoint inhibitor is in a solution at a concentration of between 1 mg/mL and 45 mg/mL, between 1 mg/mL and 40 mg/mL, between 1 mg/mL and 35 mg/mL, between 1 mg/mL and 30 mg/mL, between 1 mg/mL and 25 mg/mL, between 1 mg/mL and 20 mg/mL, between 1 mg/mL and 15 mg/mL, between 1 mg/mL and 10 mg/mL, or between 1 mg/mL and 5 mg/mL. In some embodiments, the checkpoint inhibitor is in a solution at a concentration of between 1 mg/mL and 10 mg/mL.

In some embodiments, the checkpoint inhibitor is administered by subcutaneous injection. In some embodiments, a SNA disclosed herein is administered intratumorally to a cutaneous solid tumor, intratumorally to a cutaneous tumor lesion, intratumorally to a cutaneous target lesion, intratumorally to a subcutaneous solid tumor, subcutaneous tumor lesion, intratumorally to a subcutaneous target lesion, intratumorally to a nodal solid tumor, intratumorally to a nodal tumor lesion, or intratumorally to a nodal target lesion.

In some embodiments, a SNA disclosed herein, or a population of SNAs disclosed herein, has or have a mean diameter of about 10 to about 150 nm. In some embodiments, the mean diameter of the SNA is from about 15 nm to about 100 nm, about 20 nm to about 100 nm, about 25 nm to about 100 nm, about 15 nm to about 50 nm, about 20 nm to about 50 nm, about 10 nm to about 70 nm, about 15 nm to about 70 nm about 20 nm to about 70 nm, about 10 nm to about 30 nm, about 15 nm to about 30 nm, about 20 nm to about 30 nm, about 10 nm to about 40 nm, about 15 nm to about 40 nm, about 20 nm to about 40 nm, about 10 nm to about 80 nm, about 15 nm to about 80 nm, or about 20 nm to about 80 nm.

In some embodiments, a population of SNAs have a mean diameter of about 10 to about 150 nm. In some embodiments, the mean diameter of the SNA is from about 15 nm to about 100 nm, about 20 nm to about 100 nm, about 25 nm to about 100 nm, about 15 nm to about 50 nm, about 20 nm to about 50 nm, about 10 nm to about 70 nm, about 15 nm to about 70 nm about 20 nm to about 70 nm, about 10 nm to about 30 nm, about 15 nm to about 30 nm, about 20 nm to about 30 nm, about 10 nm to about 40 nm, about 15 nm to about 40 nm, about 20 nm to about 40 nm, about 10 nm to about 80 nm, about 15 nm to about 80 nm, or about 20 nm to about 80 nm.

In some embodiments, the terms “liposomal core” and “liposome core” are used interchangeably. In some embodiments, the core (e.g., a liposomal core or liposome core) of a SNA disclosed herein, or the cores (e.g., a liposomal core or liposome core) of a population of SNAs disclosed herein, has or have a mean diameter of about 10 to about 150 nm. In some embodiments, the mean diameter of the core is from about 15 nm to about 100 nm, about 20 nm to about 100 nm, about 25 nm to about 100 nm, about 15 nm to about 50 nm, about 20 nm to about 50 nm, about 10 nm to about 70 nm, about 15 nm to about 70 nm, about 20 nm to about 70 nm, about 10 nm to about 30 nm, about 15 nm to about 30 nm, about 20 nm to about 30 nm, about 10 nm to about 40 nm, about 15 nm to about 40 nm, about 20 nm to about 40 nm, about 10 nm to about 80 nm, about 15 nm to about 80 nm, or about 20 nm to about 80 nm.

In some embodiments, the core (e.g., a liposomal core or liposome core) of a SNA disclosed herein, or the cores (e.g., a liposomal core or liposome core) of a population of SNAs disclosed herein, has or have a mean diameter of about or less than about 10 nm, 15 nm, 20 nm, 25 nm, 30 nm, 35 nm, and/or 40 nm, or any range or combination thereof.

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

In order that the invention described herein may be more fully understood, the following examples are set forth. The examples described in this application are offered to illustrate the compounds, pharmaceutical compositions, and methods provided herein and are not to be construed in any way as limiting their scope.

### EXAMPLES

### Example 1. Synopsis

CpG7909-SNA is a spherical nucleic acid (SNA) configuration of a toll-like receptor (TLR) 9 agonist oligonucleotide comprising the nucleotide sequence: 5′-T*C*G*T*C*G*T*T*T*T*G*T*C*G*T*T*T*T*G*T*C*G*T*T-3′/HEG/HEG/TEG Cholesteryl Ester/(SEQ ID NO: 1), wherein the C is 2′-deoxy-P-cytidylyl, G is 2′-deoxy-P-guanylyl, T is 2′-deoxy-P-thymidylyl, HEG is Hexa(ethylene glycol)phosphodiester, TEG Cholesteryl Ester is (N-cholesteryl-3-aminopropyl)-triethyleneglycol-glyceryl-1-O-phosphodiester and * represents a phosphorothioate internucleotide linkage.

CpG7909-SNA is a medicinal product for the treatment of advanced solid tumors. CpG-SNA exhibits a suite of mechanistic and functional properties that make it ideal for agonizing TLR9. First, SNAs are more efficiently taken into and concentrated in endosomes compared to linear oligonucleotides (i.e., oligonucleotides not in the SNA configuration). Second, the oligonucleotides delivered as a part of SNAs cause an enhanced cytokine response, both in magnitude and duration, compared to linear oligonucleotides. Third, the SNA projects its oligonucleotides outward, allowing it to act upon TLRs directly. This arrangement of nucleic acids in the SNA is in contrast to other oligonucleotide delivery systems where the oligonucleotides are held internally, for example, inside virus-like particles.

In preclinical mouse models of anti-PD-1-resistant tumors, such as the breast EMT-6 model, anti-PD-1 antibodies showed no activity, whereas the combination of CpG-SNA and anti-PD-1 antibodies demonstrated significant tumor growth inhibition and prolonged mouse survival. Further, CpG-SNA outperformed linear oligonucleotides at the same dose.

Four dose levels of CpG7909-SNA have been explored in a phase 1a first-in-human healthy volunteer study. Single doses were administered at 5-18.8m/kg subcutaneously (SC). There were no dose limiting toxicities (DLTs) and no serious adverse events (SAEs) in the study. CpG-SNA was undetectable in serum at all dose levels tested.

### Dosage and Administration

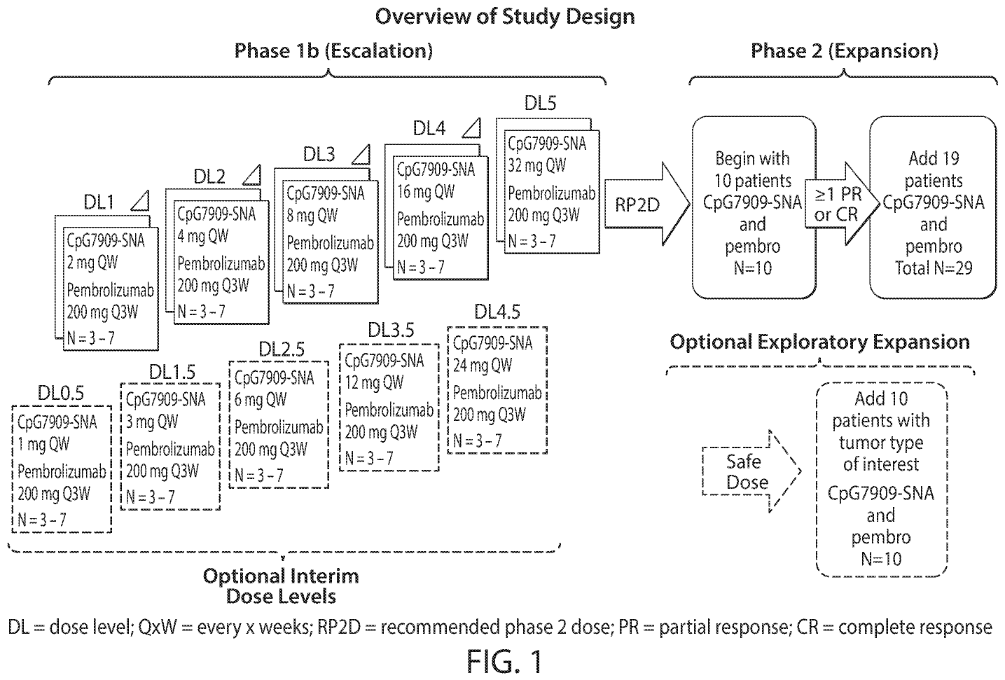

CpG7909-SNA will be administered intratumorally (IT) into a maximum of 4 accessible lesions per administration, which are amenable to repeat administration, in cohorts of escalating flat doses of 2, 4, 8, 16, and 32 mg starting on day 1 of cycle 1. One witness lesion must remain un-injected throughout the study. Injection of deep visceral lesions is not permitted, but these may be considered target lesions for efficacy evaluation. Intermediate/de-escalation dose levels such as 1, 3, 6, 12, and 24 mg may also be explored (see FIG. 1).

Cycle 1 (CpG7909-SNA monotherapy) will be 2 weeks long and comprise IT injections on days 1 and 8. All other cycles will be 3 weeks long. For cycles 2 and 3, CpG7909-SNA will be administered on a weekly basis, on days 1, 8, and 15 of each 3-week cycle. Starting at cycle 4, CpG7909-SNA will be administered every three weeks starting on day 1 until lack of clinical benefit or disease progression.

Pembrolizumab will be administered at a dose of 200 mg as an intravenous (IV) infusion over 30 minutes, on the same working day as the CpG7909-SNA injection, every 3 weeks starting at cycle 2 day 1, as per the USPI [Keytruda® USPI].

### Study Design

The study is a classical 3+3 design, ascending dose, phase 1b study of CpG7909-SNA combined with pembrolizumab in cancer patients. Patients will be dosed twice with CpG7909-SNA as a monotherapy before adding pembrolizumab, which will be added starting at the second cycle. FIG. 1 shows the overall study design.

### Example 2. Dose, Route, Dose Increments, and Flat Dosing

The starting dose of CpG7909-SNA for this trial in advanced cancer patients is 2 mg/week (equivalent to 0.031 mg/kg/week for a 65-kg patient). The systemic and local no-observed-adverse-effect levels (NOAEL(s)) defined in the 5-week monkey study using SC administration with weekly Cp7909G-SNA dosing were 3.6 and 1.2 mg/kg/week, respectively, which are 116-fold and 39-fold higher, respectively, than the proposed starting dose level. Similarly, for the 5-week rat study with weekly CpG7909-SNA dosing, the overall NOAEL for SC was 4.5 mg/kg/week, which is 145-fold higher than the proposed starting dose level.

Importantly, it is appropriate to extrapolate the NOAEL dose levels in animals on a mg/kg basis rather than applying body surface area-based (BSA-based) scaling, because the latter paradigm is suitable only to cytotoxic anticancer agents that are metabolized much more quickly than CpG7909-SNA, mainly by the cytochrome P450 system, in rodents vs. higher species. In contrast to the species differences in NOAELs observed for cytotoxics, which give much higher NOAELs in rodents, oligonucleotides typically exhibit similar or lower NOAELs in rodents vs. higher species. For the CpG7909-SNA program, the NOAEL in rats was only slightly higher than in monkeys. Essentially, there are no substantial differences in the toxicity between rats and monkeys administered CpG7909-SNA, which undermines support for BSA-based scaling and points towards the use of body weight-based extrapolation to humans. Furthermore, the area under the plasma concentration-time curve (AUC) values for rats given 1 mg/kg/week were approximately 2-fold greater than the AUC for monkeys given a similar dose level of 0.75 mg/kg/week, which again is inconsistent with the pattern observed for cytotoxic anticancer agents where the drug is cleared much more quickly by rodents, thereby further strengthening the view that BSA-based scaling is irrelevant for determining the safety margin for CpG7909-SNA. Application of BSA-based scaling to define the human equivalent doses would falsely reflect a lesser margin of safety for the proposed starting dose level and/or dictate a starting dose level that is below the pharmacologic range for CpG7909-SNA, which is considered suboptimal in cancer patients.

The proposed design uses IT dosing of CpG7909-SNA to achieve high drug levels at the target site. There are no drug disposition data from animals given IT injections of CpG7909SNA. However, in the proposed clinical study, the CpG7909-SNA will be injected into palpable or superficial, mainly subcutaneous tumors, so the absorption and distribution of drug is expected to conform reasonably closely to what was characterized with SC dosing in animals and humans. In addition, several TLR9 agonists have been administered IT with no unexpected toxicity increases compared to prior SC administration, apart from local AEs caused by the procedure itself [Diab 2017, Milhem 2018, Ribas 2018].

The only clinical study conducted to date with CpG-SNA has been a first-in-human phase 1a study (CpG7909-SNA) in healthy volunteers. Four dose levels of CpG7909-SNA, at 5, 10, 12.5, and 18.8 μg/kg have been evaluated. Each completed cohort included four subjects, and all received a single dose of CpG7909-SNA SC, for a total of 16 subjects exposed. The highest total single dose of CpG7909-SNA to any subject has been 1.4 mg. Adverse events (AEs) observed were generally mild to moderate, and no DLTs or SAEs have been reported. All AEs resolved. The expected PD effects were seen at all dose levels, while PK was below the lower limit of detection, again as expected for a TLR9 agonist given SC at low dose levels.

The pharmacophore of CpG7909-SNA drug substance is identical to CpG-7909, which is an immunostimulatory oligonucleotide that was developed for non-small cell lung cancer and other indications through phase 3 trials. At least 12 clinical studies have been conducted with CpG-7909 (not in the SNA format) [Kreig 2012]. The highest dose of CpG-7909 reported in the literature is 0.81 mg/kg, corresponding to a dose of 53 mg in a 65 kg patient [Thompson 2009]. In this study, 40 patients were dosed and an MTD was not reached. One patient who received 0.54 mg/kg had DLTs of G3 non-hematologic AEs, including anorexia. The most commonly reported AEs were flu-like symptoms and local injection-site reactions of mild-to-moderate severity. The most commonly reported serious AE was G3 fatigue in 4 patients (10%). Grade 4 AEs included anemia, exacerbated dyspnea, and polyarthralgia in 1 patient each. CpG-7909 development was discontinued for lack of efficacy. Indeed, simply activating the innate immune system is insufficient to produce an anti-tumor response. Immune checkpoint inhibition is also needed to prevent the tumor from evading the immune response.

A flat dosing schedule is described in the current study, which represents a change from the phase 1a study. In the phase 1a study of CpG7909-SNA in healthy volunteers (CpG7909-SNA), the concentration of 12 circulating cytokines was measured at multiple timepoints after the administration of CpG7909-SNA. An analysis of expression of a number of the individual cytokines vs. the dose denominated in mg/kg or the flat dose in mg revealed, quite surprisingly, that the flat dose was more predictive of the peak cytokine concentration observed in the subject plasma. Further, flat doses more accurately predicted the peak NK and pDC cell activation when compared to body mass denominated doses.

### Monotherapy DLT Period

A 2-week DLT period for the monotherapy portion of the study is justified based on the AE and PD data from the CpG7909-SNA-101 study, which show that AEs and PD effects of CpG7909-SNA resolve by day 5, suggesting that weekly dosing is acceptable. In addition, two administrations of CpG7909-SNA can be performed in a 2-week period, to allow for detection of potential cumulative effects, although these have not been seen with either the pharmacophore or with other TLR9 agonists. The PK of other TLR9 agonists (short maximum observed plasma concentration [Cmax] and terminal elimination half-life [t^{1/2}] with recovery to baseline levels by 24 hours) also supports a 2-week DLT period for the monotherapy.

Good tolerability was seen in the phase 1a healthy volunteer study, with no DLTs or SAEs (see Table 1 below).

TABLE 1
Percent of related AEs in a healthy volunteer phase 1a single SC dose study

| | CpG7909-SNA Dose Level | | | | | | | | | | | |
|---|---|---|---|---|---|---|---|---|---|---|---|---|
| | 18.8 μg/kg | | | | 12.5 μg/kg | | | 10 μg/kg | | 5 μg/kg | | |
| CTCAE V4.03 worst | (n = 4) | | | | (n = 4) | | | (n = 4) | | (n = 4) | | All |
| grade, % subjects | G1 | G2 | G3 | G4 | G1 | G2 | G3 | G1 | G2 | G1 | G2 | (n = 16) |
| Any AE | 100 | 25 | 25 | 25 | 100 | 50 | 25 | 100 | 50 | 100 | 50 | 100 |
| Flu-like symptoms | 100 | — | — | — | — | 25 | — | 75 | 25 | — | — | 56 |
| Temperature* | 100 | — | — | — | — | 25 | — | 50 | 25 | — | — | 50 |
| Sinus tachycardia* | — | — | — | — | — | — | — | 25 | — | — | — | 6 |
| Injection site reaction | 100 | — | — | — | 100 | — | — | 100 | — | 100 | — | 100 |
| Lymphadenopathy | 100 | — | — | — | 75 | — | — | 25 | — | 100 | — | 75 |
| Neutropenia | — | 25 | — | 25 | — | 50 | — | 25 | 25 | 50 | 50 | 63 |
| Lymphopenia | 75 | — | 25 | — | 50 | 25 | 25 | 25 | 50 | — | 50 | 81 |
| Muscle twitching | — | — | — | — | — | — | — | — | — | 25 | — | 6 |
| Eye pain | — | — | — | — | — | — | — | — | — | 25 | — | 6 |
| Headache | — | — | — | — | — | — | — | — | — | 25 | — | 6 |
| Hyperesthesia | 25 | — | — | — | — | — | — | — | — | — | — | 6 |

### Example 3. Description of Overall Method

An open-label, two-part, phase 1b/2 dose-finding study designed to determine the safety, tolerability, pharmacokinetics (PK), pharmacodynamics (PD), and proof-of-concept efficacy of IT CpG-SNA alone and in combination with pembrolizumab in patients with advanced solid tumors considered amenable to anti-PD-1 therapy is provided. The study consists of a dose-escalation phase followed by dose expansion.

FIG. 1 shows the overall study design, including planned dose escalation cohorts. The study uses a classical 3+3 dose escalation design, with ascending doses of CpG7909-SNA and enrollment of 3 patients per cohort and expansion to 6 patients in the event of a DLT. There will be two DLT periods for each patient: a monotherapy DLT period (15 days) and a combination DLT period of 22 days. AEs will be assessed per the Common Terminology Criteria for Adverse Events (CTCAE) v5.0, except for cytokine release syndrome, which will have its own grading system per Lee 2014.

Patients must have at least two target lesions evaluable per Response Evaluation Criteria in Solid Tumors (RECIST) 1.1. Patients must agree to provide a newly obtained biopsy of injected and witness lesions (that can be biopsied based on the investigator's assessment) prior to starting study treatment, and to repeat biopsies twice during study treatment, and to providing the acquired tissue for biomarker analysis. One witness lesion must remain un-injected throughout the study.

Patients will be dosed twice with CpG7909-SNA as a monotherapy before adding pembrolizumab, which will be added starting at the second cycle. Once the MTD or highest escalation cohort has been reached, or notable efficacy has been observed at a given dose level, and a decision as to recommended phase 2 dose (RP2D) has been taken, a two-stage expansion cohort will be initiated in patients with advanced solid tumors.

The study is a 3+3 design, ascending dose, phase 1b study of IT CpG7909-SNA combined with pembrolizumab in cancer patients. There will be two DLT periods for each patient: a monotherapy DLT period of 15 days (Cycle 1) and a combination DLT period of 22 days (Cycle 2).

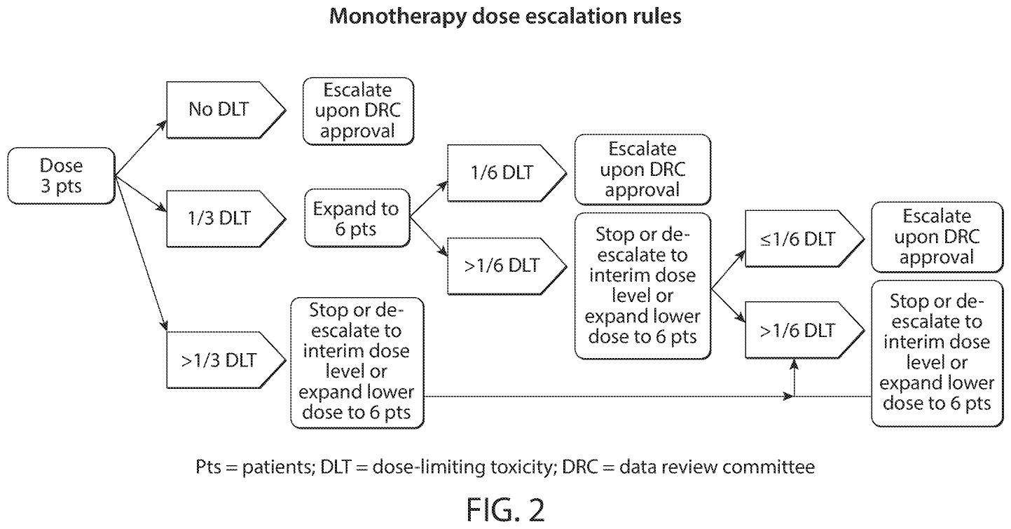

Each escalation cohort will first recruit one patient to receive CpG7909-SNA. After 7 days, the patient's AEs and lab values will be assessed and, in the absence of DLT, that patient will continue with another weekly dose of CpG7909-SNA to complete the 15-day monotherapy DLT period. At the time the first patient progresses to their second IT injection of CpG7909-SNA, two additional patients can be recruited to the cohort. Therefore, patients 2 and 3 can be dosed starting on day 8 of the first treated patient. In addition, if 6 patients are required in a cohort there is no delay required for those patients to enter the study. These patients will also be assessed for early toxicity at day 7, prior to their second doses of CpG-SNA. Once a patient has received 15 days of therapy, in the absence of DLT, combination therapy may begin for that patient. Once enough patients receive CpG-SNA monotherapy (i.e., three patients in the absence of any DLT and six in the event of one DLT), dose escalation may proceed to a new cohort upon agreement of the data review committee (DRC). See FIG. 2 for the monotherapy dose escalation rules.

The DLT definitions are provided in Table 11. For consideration of the MTD, any potential autoimmune AE≥G2 or any chronic G≥2 toxicity thought to be related to study drug(s) will also be considered.

During the monotherapy portion of the escalation, if 1/3 patients experience DLT, the cohort will expand to six patients. If the cohort expansion results indicate that 1/6 patients have DLT, escalation may proceed. If ≥2/6 patients have DLT, and if only three patients were evaluated in the dose cohort below, the cohort below will be expanded to six patients. Alternatively, if >1 DLT is seen at any dose level, the DRC may decide to deescalate to an intermediate dose level, such as 24, 12, 6, 3, or 1 mg in an optional interim dose level if deemed appropriate. Planned and optional dose levels are in presented in Table 2 and Table 3.

TABLE 2
Planned dose levels

| Planned | Planned |
|---|---|
| CpG7909-SNA Dose Level | CpG7909-SNA Dose (mg) |
| 1 | 2 |
| 2 | 4 |
| 3 | 8 |
| 4 | 16 |
| 5 | 32 |

TABLE 3
Optional interim dose levels

| Optional Interim | |
|---|---|
| CpG7909-SNA Dose level | CpG7909-SNA Dose (mg) ^{a} |
| 0.5 | 1 |
| 1.5 | 3 |
| 2.5 | 6 |
| 3.5 | 12 |
| 4.5 | 24 |

^{a} The interim CpG7909-SNA dose level may be evaluated if CpG7909-SNA is not tolerated at a planned dose. The interim CpG7909-SNA dose will be between the intolerable dose and the previous planned dose level.

If the maximum tolerated dose (MTD) is identified with inclusion of only 3 patients (e.g., the first dose level has >1 DLT and the de-escalation dose level has 0 DLTs), a further 3 patients should be enrolled to confirm safety prior to starting the expansion cohort. Once all feasible cohorts have been explored, AE and lab data from further cycles of treatment outside the DLT period will be considered in the selection of the RP2D/MTD, with particular attention paid to potential autoimmune effects, late toxicities seen in the 90-day follow-up period, and the relative dose intensity of CpG7909-SNA and pembrolizumab (meaning actual/planned dose per cycle).

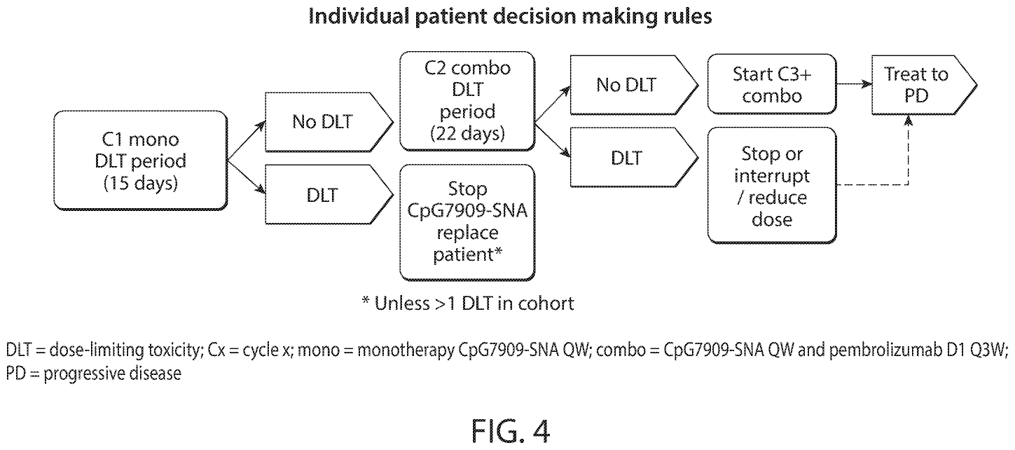

Any patients who experience DLT on CpG7909-SNA alone will discontinue therapy and not receive pembrolizumab. Because six patients will be required for the assessment of combination therapy DLTs, if one patient experiences monotherapy DLT, another patient will be added and the cohort will be expanded to a total of 7 patients (see FIG. 4). After day 15, patients will receive weekly CpG7909-SNA doses for the next two cycles, and pembrolizumab every three weeks until disease progression/lack of clinical benefit or discontinuation for AEs. For cycles four and beyond, both CpG7909-SNA and pembrolizumab will be administered on day 1 of each cycle.

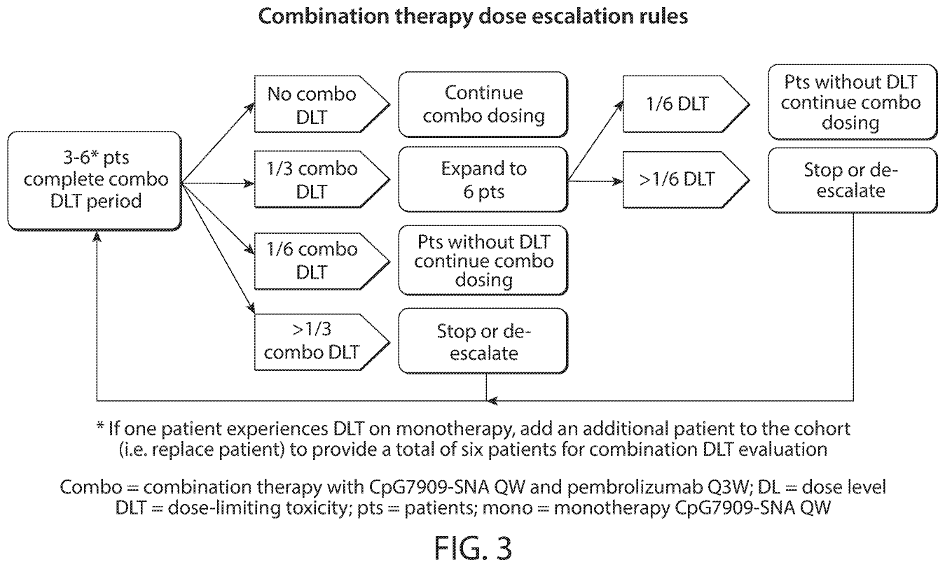

Patients will be assessed for DLTs during their first cycle of combination therapy and the same rules will be applied for de-escalation (see FIG. 3). If an MTD is identified with inclusion of only 3 patients, a further 3 patients should be enrolled to confirm safety prior to starting the expansion cohort.

If a suspected DLT occurs, a DRC meeting will be held as quickly as possible to make a judgement on the occurrence of the DLT. In the meantime, dosing of the ongoing patients in that cohort, including the dosing of pembrolizumab, will continue unless there is reason to suspect there is an unacceptable safety risk based on the nature and/or severity of the observed DLT.

If an individual patient experiences DLT in combination with pembrolizumab (Cycle 2), that patient will discontinue the study drug, unless that DLT can be managed per the Keytruda® label and the benefit is thought to outweigh the risk for that individual (see FIG. 4).

In this study, a CpG-SNA DLT is defined and occurring within 15 days of starting treatment together with a reasonable chance the AEs are related to the study drug based upon the determination of the investigator (and subsequently the DRC).

A combination DLT is defined as above, but the AE relationship may be to either study drug. The combination DLT period is from days 1 to 22 of cycle two.

### Example 4. Progress in Patients with Solid Tumors and Initial Efficacy in Patients with Merkel Cell Carcinoma

Phase 1b/2 dose escalation shows safety and tolerability. There are preliminary signs of efficacy observed in Merkel Cell Carcinoma (MCC) patients previously refractory to checkpoint inhibitor therapy. Phase 1b/2 clinical trial in patients with solid tumors evaluates AST-008, which is an SNA consisting of TLR9 agonist designed for immuno-oncology application, in combination with pembrolizumab in patients previously refractory to checkpoint inhibitors. The primary objective of the dose escalation portion of the study is to evaluate the safety, tolerability, PK, and PD of AST-008 alone and in combination with pembrolizumab, and to produce a recommended Phase 2 dose. No treatment-related serious adverse events (SAEs) or dose limiting toxicities (DLTs) have been observed. Fourteen patients have been enrolled and dosed with AST-008. The final planned dose cohort is being enrolled.

The study has enrolled five melanoma patients, four MCC patients, two cutaneous squamous cell carcinoma (CSCC) patients, two head and neck squamous patients, and one mucosal melanoma patient. Most patients had progressive disease on anti-PD-(L)1 antibodies prior to enrolling.

The data demonstrate that AST-008 administration alone or in combination with pembrolizumab produces cytokine and chemokine expression and immune cell activation in patient blood indicative of desired immune activation. Furthermore, one MCC patient that previously progressed on anti-PD-1 antibody therapy has confirmed stable disease in excess of twelve weeks with decreased target lesion diameters, while a second MCC patient experienced a target lesion complete response and a confirmed overall partial response with over 24 weeks duration. Among the additional patients already enrolled, two have yet to be evaluated for efficacy, one was not evaluable, and the remaining patients had progressive disease. The initial responses observed in patients is highly encouraging, given that no second line therapies are approved for patients with MCC. Any responses seen in these patient populations is significant. Based on these early results showing positive biomarker data and initial responses, patients resistant to anti PD-1/PD-L1 therapy are to be investigated in a Phase 2 study in MCC and in cutaneous squamous cell carcinoma (CSCC).

### REFERENCES

Aryan Z, Holgate S T, Radzioch D, et al. A new era of targeting the ancient gatekeepers of the immune system: toll-like agonists in the treatment of allergic rhinitis and asthma. International Archives of Allergy and Immunology. 2014; 164, 46-63. Cohen E, Milhem M, Ribas A, et al. Phase 1b/2, Open-Label, Multicenter Study of Intratumoral SD-101 in Combination with Pembrolizumab in Anti-PD-1 Treatment-Naïve Patients with Recurrent or Metastatic Head and Neck Squamous Cell Carcinoma. Presented at: 2018 American Association for Cancer Research (AACR) Annual Meeting; Apr. 14-18, 2018; Chicago, Ill. Abstract CT098.
Diab A, Haymaker C, Uemura M, et al. A Phase 1/2 trial of intratumoral (i.t.) IMO-2125 (IMO) in combination with checkpoint inhibitors (CPI) in PD-(L)1-refractory melanoma. Ann Oncol 2017; 28(suppl_5):v403-v427.
Eisenhauer E A, Therasse P, Bogaerts J, et al. 2009. New response evaluation criteria in solid tumours: Revised RECIST guideline (version 1.1). Eur J Cancer. 45(2):228-247.
Keytruda® United States Prescribing Information 2018
Krieg A M. CpG still rocks! Update on an accidental drug. Nucleic Acid Ther. 2012; 22, 77-89.
Libtayo® United States Prescribing Information 2019.
Lee D W, Gardner R, Porter D L, et al. Current concepts in the diagnosis and management of cytokine release syndrome. Blood 2014; 124(2):188-195.
Lentz H J. 2007. Management and preparedness for infusion and hypersensitivity reactions. Oncologist. 12:601-609.
Levey A S, Stevens L A, Schmid C H, et al. A new equation to estimate glomerular filtration rate. Ann Intern Med. 2009; 150(9):604-612.
Milhem M, Gonzales R, Medina T, et al. Intratumoral toll-like receptor 9 (TLR9) agonist, CMP-001, in combination with pembrolizumab can reverse resistance to PD-1 inhibition in a phase 1b trial in subjects with advanced melanoma. Presented at: AACR Annual Meeting 2018; Apr. 14-18, 2018; Chicago, Ill. Abstract CT0101.
Nallagatla S R, Anderson B R, Kang R, et al. Abstract 3758: TLR9 agonist SNA-induced innate and adaptive immune responses in tumor microenvironment enhance checkpoint inhibitor antitumor activity in mouse tumor models. AACR Annual Meeting 2018; Apr. 14-18, 2018; Chicago, Ill.
Opdivo® United States Prescribing Information 2018
Poh A. Warming “cold” melanoma with TLR9 agonists. Cancer Discov. 2018a June; 8(6):670. doi: 10.1158/2159-8290.CD-ND2018-004. Epub 2018 Apr. 18. Ribas A, Medina T, Kummar S, et al. Durability of Responses to SD-101 in Combination with Pembrolizumab in Advanced Metastatic Melanoma: Results of a Phase 1b, Multicenter Study. Presented at: 2018 American Association for Cancer Research (AACR) Annual Meeting; Apr. 14-18, 2018b; Chicago, Ill. Abstract CT139.
Sampson H A, Muñoz-Furlong A, Campbell R L, et al. Second symposium on the definition and management of anaphylaxis: summary report—Second National Institute of Allergy and Infectious Disease/Food Allergy and Anaphylaxis Network symposium. J Allergy Clin Immunol. 2006; 117(2):391-7. Sato-Kaneko F, Yao S, Ahmadi A, et al. Combination immunotherapy with TLR agonists and checkpoint inhibitors suppresses head and neck cancer. JCI Insight. 2017; 2(18):e93397. https://doi.org/10.1172/jci.insight.93397. Schmidt M, Kapp K, Volz B et al. Combination of TLR9 agonist lefitolimod/MGN1703 with checkpoint inhibitors for cancer immunotherapy. J Clin Oncol 2017; 35 (4_suppl):a634.
Simon R, Designs for efficient clinical trials. Oncology (Williston Park) 1989; 3(7):43-9; discussion 51-3.
Thompson J A, Kuzel T, Drucker B J, et al. Safety and efficacy of PF-3512676 for the treatment of stage IV renal cell carcinoma: an open-label, multicenter phase I/II study. Clinical Genitourinary Cancer. 2009; 7,E58-65.
Wang S, Campos J, Naik E, et al. Intratumoral Treatment With A Highly Interferogenic TLR9 Agonist Reverts Tumor Escape From PD-1 Blockade. [Abstract]. In: Proceedings Of The 107th Annual Meeting Of The American Association For Cancer Research; 2016 Apr. 16-20; New Orleans, La. Philadelphia (PA): AACR; Cancer Res 2016;76(14 Suppl):Abstract Nr 2322.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

### Other Embodiments

Embodiment 1. A method for treating cancer comprising:
   administering to a subject a spherical nucleic acid (SNA) and a checkpoint inhibitor,
   wherein the SNA comprises a core having an oligonucleotide shell comprised of CpG oligonucleotides positioned on the exterior of the core, wherein the SNA is administered to the subject at a fixed dose of at least about 2 mg that is divided among two or more solid tumors or tumor lesions in the subject,
   wherein the SNA is administered within 24 hours of administration of the checkpoint inhibitor, to treat the cancer in the subject.
Embodiment 2. A method for treating cancer comprising:
   administering to a subject a spherical nucleic acid (SNA) and a checkpoint inhibitor,
   wherein the SNA comprises a core having an oligonucleotide shell comprised of CpG oligonucleotides positioned on the exterior of the core, wherein the SNA is administered at a dose of between 2 mg and 32 mg every three weeks,
   wherein the checkpoint inhibitor is administered at a dose of between 180 and 220 mg every three weeks,
   wherein the SNA is administered within 24 hours of the administration of the checkpoint inhibitor, and
   wherein the SNA and the checkpoint inhibitor are administered through different routes of administration to treat the cancer in the subject.
Embodiment 3. A method of treating cancer comprising:
   administering a therapeutic dose of a spherical nucleic acid (SNA) comprising a CpG oligonucleotide linked through a spacer to an exterior surface of a liposome core having a mean diameter of less than 40 nm and a checkpoint inhibitor, wherein the SNA is administered by intratumoral injection into multiple lesions at a dose of between 2 mg and 32 mg and the checkpoint inhibitor is administered by intravenous injection at a dose of between 180 and 220 mg.
Embodiment 4. The method of any one of embodiments 1-3, wherein the cancer is biliary tract cancer, brain cancer, breast cancer, cervical cancer, choriocarcinoma, colon cancer, endometrial cancer, esophageal cancer, gastric cancer, an intraepithelial neoplasm, leukemia, lymphoma, liver cancer, lung cancer, melanoma, neuroblastoma, oral cancer, ovarian cancer, pancreatic cancer, pancreatic adenocarcinoma, prostate cancer, hormone refractory prostate adenocarcinoma, rectal cancer, sarcomas, testicular cancer, thyroid cancer, anaplastic thyroid cancer, renal cancer, hairy cell leukemia, chronic myelogenous leukemia, cutaneous T-cell leukemia, multiple myeloma, renal cell carcinoma, clear cell renal cell carcinoma, lymphoma, bladder cancer, non-small cell lung cancer (NSCLC), or glioma, glioblastoma multiforme.
Embodiment 5. The method of any of embodiments 1-4, wherein the spacer is oligoethylene glycol.
Embodiment 6. The method of any one of embodiments 1-5, wherein the checkpoint inhibitor is a PD-1 antibody.
Embodiment 7. The method of any one of embodiments 1-6, wherein the checkpoint inhibitor is pembrolizumab.
Embodiment 8. A method for treating cancer comprising:
   administering to a subject a spherical nucleic acid (SNA) and a checkpoint inhibitor,
   wherein the SNA comprises a core having an oligonucleotide shell comprised of CpG oligonucleotides positioned on the exterior of the core, wherein the SNA is administered at a dose of between about 16 mg to about 32 mg every three weeks,
   wherein the checkpoint inhibitor is administered at a dose of between 180 and 220 mg every three weeks,
   wherein the SNA is administered within 24 hours of the administration of the checkpoint inhibitor, and
   wherein the SNA and the checkpoint inhibitor are administered through different routes of administration to treat the cancer in the subject, wherein the cancer is Merkel cell carcinoma or cutaneous squamous cell carcinoma.
Embodiment 9. A method of treating cancer comprising:
   administering a therapeutic dose of a spherical nucleic acid (SNA) comprising a CpG oligonucleotide linked through a spacer to an exterior surface of a liposome core having a diameter of less than about 40 nm and a checkpoint inhibitor, wherein the SNA is administered by intratumoral injection into multiple lesions at a dose of between about 16 mg and about 32 mg and the checkpoint inhibitor is administered by intravenous injection at a dose of between 180 and 220 mg, wherein the cancer is Merkel cell carcinoma or cutaneous squamous cell carcinoma.
Embodiment 10. The method of any one of embodiments 1-9, wherein administration of the SNA or the SNA in combination with the checkpoint inhibitor results in one or more of increased cytokine expression, increased chemokine expression, or increased immune cell activation by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, relative to a reference level.

1. A method for treating cancer comprising:
administering to a subject a spherical nucleic acid (SNA) and a checkpoint inhibitor,
wherein the SNA comprises a core having an oligonucleotide shell comprised of CpG oligonucleotides positioned on the exterior of the core, wherein the SNA is administered to the subject at a fixed dose of at least about 2 mg to one solid tumor or tumor lesion or divided among two or more solid tumors or tumor lesions in the subject,
wherein the SNA is administered within 24 hours of administration of the checkpoint inhibitor, to treat the cancer in the subject.

2. A method for treating cancer comprising:
administering to a subject a spherical nucleic acid (SNA) and a checkpoint inhibitor,
wherein the SNA comprises a core having an oligonucleotide shell comprised of CpG oligonucleotides positioned on the exterior of the core, wherein the SNA is administered at a dose of between 2 mg and 32 mg once a week or every three weeks,
wherein the checkpoint inhibitor is administered at a dose of between 180 and 370 mg every three weeks, or at a dose of between 700 mg and 900 mg every two weeks,
wherein the SNA is administered within 24 hours of the administration of the checkpoint inhibitor, and
wherein the SNA and the checkpoint inhibitor are administered through different routes of administration to treat the cancer in the subject.

3. A method for treating cancer comprising:
administering a therapeutic dose of a checkpoint inhibitor and a therapeutic dose of a spherical nucleic acid (SNA) comprising a CpG oligonucleotide linked through a spacer to an exterior surface of a liposome core having a mean diameter of less than 40 nm, wherein the SNA is administered by intratumoral injection into one tumor lesion or into multiple lesions at a dose of between 2 mg and 32 mg and wherein the checkpoint inhibitor is administered by intravenous injection at a dose of between 180 and 800 mg.

4. The method of claim 1 or 2, wherein the SNA is administered subcutaneously or intratumorally to a solid tumor and wherein the checkpoint inhibitor is administered by intravenous infusion.

5. The method of any one of claims 1-4, wherein the cancer in the subject is not responsive to treatment with the checkpoint inhibitor alone or wherein the cancer in the subject is resistant to treatment with the checkpoint inhibitor alone.

6. The method of any one of claims 1-5, wherein the subject has not received a small molecule or tyrosine kinase inhibitor within 2 weeks or 5 half-lives (whichever is longer) prior to the first dose of the SNA, has not received chemotherapy within 3 weeks prior to the first dose of the SNA, has not received biological cancer therapy within 3 weeks prior to the first dose of the SNA, has not received nitrosourea or radioisotope within 6 weeks prior to first dose of the SNA, has not recovered from an adverse event (G1) or has not been identified as experiencing an adverse event due to cancer therapeutics administered more than 4 weeks prior to the first dose of the SNA.

7. The method of any one of claims 1-6, wherein the SNA is administered at a dose of between 1 and 3 mg.

8. The method of any one of claims 1-6, wherein the SNA is administered at a dose between 3 and 5 mg.

9. The method of any one of claims 1-6, wherein the SNA is administered at a dose between 5 and 7 mg.

10. The method of any one of claims 1-6, wherein the SNA is administered at a dose between 7 and 9 mg.

11. The method of any one of claims 1-6, wherein the SNA is administered at a dose between 9 and 14 mg.

12. The method of any one of claims 1-6, wherein the SNA is administered at a dose between 15 and 17 mg.

13. The method of any one of claims 1-6, wherein the SNA is administered at a dose between 18 mg and 31 mg.

14. The method of any one of claims 1-6, wherein the SNA is administered at a dose between 31 mg and 33 mg.

15. The method of any one of claims 1-6, wherein the SNA is administered at a dose between 0.5 mg and 2 mg.

16. The method of any one of claims 1-6, wherein the SNA is administered at a dose between 2 and 4 mg.

17. The method of any one of claims 1-6, wherein the SNA is administered at a dose between 11 and 13 mg.

18. The method of any one of claims 1-6, wherein the SNA is administered at a dose between 23 and 25 mg.

19. The method of any one of claims 1-6, wherein the SNA is administered at a dose between 2 and 31 mg.

20. The method of any one of claims 1-6, wherein the SNA is administered at a dose between 2 and 30 mg.

21. The method of any one of claims 1-6, wherein the SNA is administered at a dose between 2 and 29 mg.

22. The method of any one of claims 1-6, wherein the SNA is administered at a dose between 2 and 28 mg.

23. The method of any one of claims 1-6, wherein the SNA is administered at a dose between 2 and 27 mg.

24. The method of any one of claims 1-6, wherein the SNA is administered at a dose between 2 and 26 mg.

25. The method of any one of claims 1-6, wherein the SNA is administered at a dose between 2 and 25 mg.

26. The method of any one of claims 1-6, wherein the SNA is administered at a dose between 2 and 24 mg.

27. The method of any one of claims 1-6, wherein the SNA is administered at a dose between 2 and 23 mg.

28. The method of any one of claims 1-6, wherein the SNA is administered at a dose between 2 and 22 mg.

29. The method of any one of claims 1-6, wherein the SNA is administered at a dose between 2 and 21 mg.

30. The method of any one of claims 1-6, wherein the SNA is administered at a dose between 2 and 20 mg.

31. The method of any one of claims 1-6, wherein the SNA is administered at a dose between 2 and 19 mg.

32. The method of any one of claims 1-6, wherein the SNA is administered at a dose between 2 and 18 mg.

33. The method of any one of claims 1-6, wherein the SNA is administered at a dose between 2 and 17 mg.

34. The method of any one of claims 1-6, wherein the SNA is administered at a dose between 2 and 16 mg.

35. The method of any one of claims 1-6, wherein the SNA is administered at a dose between 2 and 15 mg.

36. The method of any one of claims 1-6, wherein the SNA is administered at a dose between 2 and 14 mg.

37. The method of any one of claims 1-6, wherein the SNA is administered at a dose between 2 and 13 mg.

38. The method of any one of claims 1-6, wherein the SNA is administered at a dose between 2 and 12 mg.

39. The method of any one of claims 1-6, wherein the SNA is administered at a dose between 2 and 11 mg.

40. The method of any one of claims 1-6, wherein the SNA is administered at a dose between 2 and 10 mg.

41. The method of any one of claims 1-6, wherein the SNA is administered at a dose between 2 and 9 mg.

42. The method of any one of claims 1-6, wherein the SNA is administered at a dose between 2 and 8 mg.

43. The method of any one of claims 1-6, wherein the SNA is administered at a dose between 2 and 7 mg.

44. The method of any one of claims 1-6, wherein the SNA is administered at a dose between 2 and 6 mg.

45. The method of any one of claims 1-6, wherein the SNA is administered at a dose between 2 and 5 mg.

46. The method of any one of claims 1-6, wherein the SNA is administered at a dose between 2 and 3 mg.

47. The method of any one of claims 1-6, wherein the SNA is administered at a dose of 1 mg.

48. The method of any one of claims 1-6, wherein the SNA is administered at a dose of 2 mg.

49. The method of any one of claims 1-6, wherein the SNA is administered at a dose of 3 mg.

50. The method of any one of claims 1-6, wherein the SNA is administered at a dose of 4 mg.

51. The method of any one of claims 1-6, wherein the SNA is administered at a dose of 6 mg.

52. The method of any one of claims 1-6, wherein the SNA is administered at a dose of 8 mg.

53. The method of any one of claims 1-6, wherein the SNA is administered at a dose of 12 mg.

54. The method of any one of claims 1-6, wherein the SNA is administered at a dose of 16 mg.

55. The method of any one of claims 1-6, wherein the SNA is administered at a dose of 24 mg.

56. The method of any one of claims 1-6, wherein the SNA is administered at a dose of 32 mg.

57. The method of any one of claims 1-56, wherein the checkpoint inhibitor is administered at a dose between 50 mg and 1000 mg.

58. The method of any one of claims 1-56, wherein the checkpoint inhibitor is administered at a dose between 50 mg and 750 mg.

59. The method of any one of claims 1-56, wherein the checkpoint inhibitor is administered at a dose between 50 mg and 500 mg.

60. The method of any one of claims 1-56, wherein the checkpoint inhibitor is administered at a dose between 50 mg and 400 mg.

61. The method of any one of claims 1-56, wherein the checkpoint inhibitor is administered at a dose between 50 mg and 300 mg.

62. The method of any one of claims 1-56, wherein the checkpoint inhibitor is administered at a dose between 50 mg and 290 mg.

63. The method of any one of claims 1-56, wherein the checkpoint inhibitor is administered at a dose between 50 mg and 280 mg.

64. The method of any one of claims 1-56, wherein the checkpoint inhibitor is administered at a dose between 50 mg and 270 mg.

65. The method of any one of claims 1-56, wherein the checkpoint inhibitor is administered at a dose between 50 mg and 260 mg.

66. The method of any one of claims 1-56, wherein the checkpoint inhibitor is administered at a dose between 50 mg and 250 mg.

67. The method of any one of claims 1-56, wherein the checkpoint inhibitor is administered at a dose between 50 mg and 240 mg.

68. The method of any one of claims 1-56, wherein the checkpoint inhibitor is administered at a dose between 50 mg and 230 mg.

69. The method of any one of claims 1-56, wherein the checkpoint inhibitor is administered at a dose between 50 mg and 220 mg.

70. The method of any one of claims 1-56, wherein the checkpoint inhibitor is administered at a dose between 50 mg and 210 mg.

71. The method of any one of claims 1-56, wherein the checkpoint inhibitor is administered at a dose between 50 mg and 200 mg.

72. The method of any one of claims 1-56, wherein the checkpoint inhibitor is administered at a dose between 60 mg and 200 mg.

73. The method of any one of claims 1-56, wherein the checkpoint inhibitor is administered at a dose between 70 mg and 200 mg.

74. The method of any one of claims 1-56, wherein the checkpoint inhibitor is administered at a dose between 80 mg and 200 mg.

75. The method of any one of claims 1-56, wherein the checkpoint inhibitor is administered at a dose between 90 mg and 200 mg.

76. The method of any one of claims 1-56, wherein the checkpoint inhibitor is administered at a dose between 100 mg and 200 mg.

77. The method of any one of claims 1-56, wherein the checkpoint inhibitor is administered at a dose between 110 mg and 200 mg.

78. The method of any one of claims 1-56, wherein the checkpoint inhibitor is administered at a dose between 120 mg and 200 mg.

79. The method of any one of claims 1-56, wherein the checkpoint inhibitor is administered at a dose between 130 mg and 200 mg.

80. The method of any one of claims 1-56, wherein the checkpoint inhibitor is administered at a dose between 140 mg and 200 mg.

81. The method of any one of claims 1-56, wherein the checkpoint inhibitor is administered at a dose between 150 mg and 200 mg.

82. The method of any one of claims 1-56, wherein the checkpoint inhibitor is administered at a dose between 160 mg and 200 mg.

83. The method of any one of claims 1-56, wherein the checkpoint inhibitor is administered at a dose between 170 mg and 200 mg.

84. The method of any one of claims 1-56, wherein the checkpoint inhibitor is administered at a dose between 180 mg and 200 mg.

85. The method of any one of claims 1-56, wherein the checkpoint inhibitor is administered at a dose between 190 mg and 200 mg.

86. The method of any one of claims 1-56, wherein the checkpoint inhibitor is administered at a dose of 200 mg.

87. The method of any one of claims 1-86, wherein the cancer is biliary tract cancer, brain cancer, breast cancer, cervical cancer, choriocarcinoma, colon cancer, endometrial cancer, esophageal cancer, gastric cancer, an intraepithelial neoplasm, leukemia, lymphoma, liver cancer, lung cancer, neuroblastoma, oral cancer, ovarian cancer, pancreatic cancer, pancreatic adenocarcinoma, prostate cancer, hormone refractory prostate adenocarcinoma, rectal cancer, sarcomas, testicular cancer, thyroid cancer, anaplastic thyroid cancer, renal cancer, hairy cell leukemia, chronic myelogenous leukemia, cutaneous T-cell leukemia, multiple myeloma, renal cell carcinoma, clear cell renal cell carcinoma, lymphoma, bladder cancer, non-small cell lung cancer (NSCLC), or glioma, glioblastoma multiforme.

88. The method of any one of claims 1-86, wherein the cancer is Merkel cell carcinoma, cutaneous squamous cell carcinoma, melanoma or squamous cell carcinoma of the head and neck.

89. The method of any one of claims 1-86, wherein the subject has a solid tumor or a tumor lesion that can be injected intratumorally via one or more of palpation or ultrasound.

90. The method of claim 89, wherein the solid tumor or tumor lesion is on or near the skin, on or near cutaneous soft tissue, on or near subcutaneous soft tissue, and/or in or near a lymph node.

91. The method of claim 89 or 90, wherein the SNA is administered to one or more of a cutaneous tumor lesion, a subcutaneous tumor lesion or a nodal lesion.

92. The method of any one of claims 1-91, wherein the liposome core is about 15 nm to 30 nm in mean diameter.

93. The method of any one of claims 1-92, wherein the CpG oligonucleotides comprise a spacer.

94. The method of claim 93, wherein the spacer is or comprises an oligoethylene glycol.

95. The method of any one of claim 94, wherein the oligoethylene glycol is a hexaethylene glycol.

96. The method of any one of claims 1-95, wherein the SNA has about 25 to 35 CpG oligonucleotides positioned on the exterior surface of the core.

97. The method of any one of claims 1-96, wherein the CpG oligonucleotides comprise the nucleotide sequence of CpG-7909.

98. The method of any one of claims 1-96, wherein the CpG oligonucleotides comprise the nucleotide sequence 5′-TCGTCGTTTTGTCGTTTTGTCGTT-3′ (SEQ ID NO: 1).

99. The method of any one of claims 1-96, wherein the CpG oligonucleotides comprise the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1).

100. The method of any one of claims 1-97, wherein the checkpoint inhibitor is a PD-1 antibody or a PD-L1 antibody.

101. The method of any one of claims 1-97, wherein the checkpoint inhibitor is pembrolizumab, avelumab, or cemiplimab.

102. The method of any one of claim 1-86 or 89-101, wherein the cancer is a sarcoma, including pleomorphic sarcoma, gastrointestinal stromal tumor (GIST), liposarcoma, leiomyosarcoma, synovial sarcoma, malignant peripheral nerve sheath tumor, rhabdomyosarcoma, angiosarcoma, fibrosarcoma, dermatofibrosarcoma protuberans, epithelioid sarcoma, myxoma, mesenchymoma, vascular sarcoma, neurilemmoma, bone sarcoma, osteosarcoma, Ewing's sarcoma, chondrosarcoma, Kaposi sarcoma, solitary fibrous tumor, chordoma, desmoid-type fibromatosis, fibroblastic sarcoma, giant cell tumor of the bone, gynaecological sarcoma, soft tissue sarcoma, angioleiomyoma, leiomyoma, smooth muscle sarcoma, fibrohistiocytic sarcoma.

103. The method of any one of claims 1-102, wherein the cancer in the subject is refractory or resistant to treatment with a checkpoint inhibitor.

104. The method of any one of claims 1-103, wherein the SNA is administered within 24 hours of administration of the checkpoint inhibitor.

105. The method of any one of claims 1-103, wherein the SNA is administered within 12 hours of administration of the checkpoint inhibitor.

106. The method of any one of claims 3-105, wherein the liposome core is less than 30 nm in mean diameter.

107. The method of any one of claims 3-105, wherein the liposome core is about 15 nm to 40 nm in mean diameter.

108. The method of any one of claim 1-103 or 107, wherein the SNA and the checkpoint inhibitor are administered substantially at the same time.

109. The method of any one of claims 1-56, wherein the checkpoint inhibitor is administered at a dose between 50 mg and 350 mg.

110. The method of any one of claims 1-56, wherein the checkpoint inhibitor is administered at a dose between 50 mg and 1200 mg.

111. The method of any one of claims 1-56, wherein the checkpoint inhibitor is administered at a dose between 1 mg/kg and 10 mg/kg.

112. The method of any one of claim 1-103, 106, 107 or 109-111, wherein the SNA is administered prior to administration of the checkpoint inhibitor.

113. The method of any one of claim 1-103, 106, 107, or 109-111, wherein the SNA is administered after the administration of the checkpoint inhibitor.

114. A method for treating cancer comprising:
administering to a subject a spherical nucleic acid (SNA) and a checkpoint inhibitor,
wherein the SNA comprises a core having an oligonucleotide shell comprised of CpG oligonucleotides positioned on the exterior of the core, wherein the SNA is administered to the subject at a fixed dose of between about 16 mg to about 32 mg to one solid tumor or tumor lesion or at a fixed dose of between about 16 mg to about 32 mg divided among two or more solid tumors or tumor lesions in the subject, wherein the SNA is administered within 24 hours of administration of the checkpoint inhibitor, to treat the cancer in the subject, wherein the cancer is Merkel cell carcinoma or cutaneous squamous cell carcinoma.

115. A method for treating cancer comprising:
administering to a subject a spherical nucleic acid (SNA) and a checkpoint inhibitor,
wherein the SNA comprises a core having an oligonucleotide shell comprised of CpG oligonucleotides positioned on the exterior of the core, wherein the SNA is administered at a dose of between about 16 mg to about 32 mg once a week,
wherein the checkpoint inhibitor is administered at a dose of between 180 mg and 370 mg every three weeks or at a dose of between 700 mg and 900 mg every two weeks,
wherein the SNA is administered within 24 hours of the administration of the checkpoint inhibitor, and
wherein the SNA and the checkpoint inhibitor are administered through different routes of administration to treat the cancer in the subject, wherein the cancer is Merkel cell carcinoma or cutaneous squamous cell carcinoma.

116. A method of treating cancer comprising:
administering a therapeutic dose of a spherical nucleic acid (SNA) comprising a CpG oligonucleotide linked through a spacer to an exterior surface of a liposome core having a diameter of less than about 40 nm and a checkpoint inhibitor, wherein the SNA is administered by intratumoral injection into multiple lesions at a dose of between about 16 mg and about 32 mg and the checkpoint inhibitor is administered by intravenous injection at a dose of between 180 and 370 mg or between 700 mg and 900 mg, wherein the cancer is Merkel cell carcinoma or cutaneous squamous cell carcinoma.

117. The method of any one of claims 114-116, wherein the SNA is administered at a dose of or about 16 mg.

118. The method of any one of claims 114-116, wherein the SNA is administered at a dose of or about 32 mg.

119. The method of any one of claims 114-118, wherein the SNA is administered at a dose of or about 16 mg and wherein the checkpoint inhibitor is administered at a dose of or about 200 mg.

120. The method of any one of claims 114-118, wherein the SNA is administered at a dose of or about 32 mg and wherein the checkpoint inhibitor is administered at a dose of or about 200 mg.

121. The method of any one of claims 114-118, wherein the checkpoint inhibitor is pembrolizumab administered at a dose of or about 200 mg and wherein the cancer is Merkel cell carcinoma.

122. The method of any one of claims 114-118, wherein the SNA is administered at a dose of or about 16 mg and wherein the checkpoint inhibitor is administered at a dose of or about 350 mg.

123. The method of any one of claims 114-118, wherein the SNA is administered at a dose of or about 32 mg and wherein the checkpoint inhibitor is administered at a dose of or about 350 mg.

124. The method of any one of claims 114-118, wherein the checkpoint inhibitor is cemiplimab administered at a dose of or about 350 mg and wherein the cancer is cutaneous squamous cell carcinoma.

125. The method of any one of claims 114-118, wherein the SNA is administered at a dose of or about 16 mg and wherein the checkpoint inhibitor is administered at a dose of or about 800 mg.

126. The method of any one of claims 114-118, wherein the SNA is administered at a dose of or about 32 mg and wherein the checkpoint inhibitor is administered at a dose of or about 800 mg.

127. The method of any one of claims 114-118, wherein the checkpoint inhibitor is avelumab administered at a dose of or about 800 mg and wherein the cancer is Merkel cell carcinoma.

128. The method of any one of claims 114-127, wherein administration of the SNA or the SNA in combination with the checkpoint inhibitor results in one or more of increased cytokine expression, increased chemokine expression, or increased immune cell activation by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, at least or about 100%, at least or about 150%, at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 15-fold, at least or about 20-fold, at least or about 30-fold, at least or about 40-fold, at least or about 50-fold or more, relative to a reference level.

129. The method of any one of claims 1-128, wherein the cancer in the subject is progressive disease and administration of the SNA or administration of the SNA in combination with the checkpoint inhibitor for the treatment of the cancer in the subject renders the cancer stable disease.

130. The method of any one of claims 1-129, wherein the cancer is stable disease for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks.

131. The method of claim 130, wherein the cancer is stable disease for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months.

132. The method of claim 130, wherein the cancer is stable disease for at least one year, at least two years, at least three years, at least four years, at least five years, at least six years, at least seven years, at least eight years, at least nine years, at least 10 years, at least 11 years or at least 12 years.

133. The method of any one of claims 1-132, wherein the subject has at least one target lesion, at least two target lesions, at least three target lesions or at least four target lesions and wherein administration of the SNA or the SNA in combination with the checkpoint inhibitor decreases the diameter of at least one target lesion in the subject or decreases the sum of the diameters of two or more target lesions in the subject by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99% relative to a reference level.

134. The method of any one of claims 1-133, wherein the subject has at least one target lesion, at least two target lesions, at least three target lesions or at least four target lesions and wherein administration of the SNA or the SNA in combination with the checkpoint inhibitor results in partial response or results in complete response in at least one target lesion, at least two target lesions, at least three target lesions, or at least four target lesions in the subject.

135. The method of any one of claims 1-134, wherein the treatment results in partial response or complete response for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks in the subject.

136. The method of any one of claims 1-134, wherein the treatment results in partial response or complete response for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months in the subject.
